(19) United States
(12) Patent Application Publication
(10) Pub. No.: US 2026/0000347 A1
(43) Pub. Date: Jan. 1, 2026

(54) SYSTEMS, APPARATUSES, AND METHODS FOR EVALUATING WORD STRUCTURES IN AUDIO RECORDINGS OF PATIENT SPEECH

(71) Applicant: SF Ambi LLC, Wilmington, DE (US)

(72) Inventor: Kevin Scott Dias, Oyama (JP)

(21) Appl. No.: 19/245,647

(22) Filed: Jun. 23, 2025

Related U.S. Application Data
(60) Provisional application No. 63/665,789, filed on Jun. 28, 2024.

(51) Int. Cl. A61B 5/00; G10L 15/02

(52) U.S. Cl. CPC A61B 5/4803; G10L 15/02; G10L 2015/025

(57) ABSTRACT

Systems, apparatuses, and methods for evaluating word structures in audio recordings are disclosed. Speech from one or more speakers may be recorded to create an audio file. The audio file may be transcribed to generate a transcript that identifies one or more words in the speech. One or more phonemes may be detected in the one or more words in the speech. The audio file may be isolated into one or more audio fragments that correspond to the one or more phonemes. The one or more audio fragments may be analyzed to determine whether the one or more phonemes in the one or more words was pronounced correctly or incorrectly in the corresponding sound-based unit in the position in the one or more words. Performance of the speech may be scored by calculating correct and incorrect pronunciations of the one or more phonemes.

### CROSS-REFERENCES TO RELATED APPLICATIONS

The present application claims priority to, and benefit from U.S. provisional patent application No. 63/665,789, filed on Jun. 28, 2024, and which is incorporated by

A portion of the disclosure of this patent document contains material that is subject to copyright protection. The copyright owner has no objection to the reproduction of the patent document or the patent disclosure, as it appears in the U.S. Patent and Trademark Office patent file or records, but otherwise reserves all copyright rights whatsoever.

### FIELD OF THE DISCLOSURE

This application relates, in general, to systems, apparatuses, and methods for evaluating word structures in audio recordings of patient speech, and more specifically, to systems, apparatuses, and methods for analyzing transcripts of patient speech to score patient performance, which can be tracked over time.

### BACKGROUND

This section is intended to introduce various aspects of the art, which may be associated with exemplary embodiments of the present disclosure. This discussion is believed to assist in providing a framework to facilitate a better understanding of particular aspects of the present disclosure. Accordingly, it should be understood that this section should be read in this light, and not necessarily as admissions of prior art.

Outside of their therapeutic services, healthcare or educational providers of various therapeutic practices are burdened with administrative tasks, such as charting and record keeping, as well as tracking the progress of their patients, clients, or students and planning for their future treatment sessions or appointments. The more time that a provider has to spend on such administrative tasks, the less time the provider may have to see a greater volume of patients. In the context of speech therapy, healthcare or educational providers are more often than not equipped with outdated or archaic technologies that facilitate the charting, recordkeeping, and audio-recording of patient-originated speech.

Healthcare or educational providers rely on outdated or archaic technologies that are limited to simple speech-to-text transcription. Simple speech-to-text transcription is insufficient for ascertaining whether a patient has correctly or incorrectly pronounced a word, or a specific sound within the word (e.g., a vowel or consonant sound) and/or in what position of the word the specific sound is located, such as an initial position, a middle position, or a tail (or final) position of the word. These technologies simply highlight whether a word was spoken so coherently (or incoherently) such that an audio file associated with the spoken word could be correctly transcribed onto a transcript. For example, prevailing speech-to-text transcription technologies fail to ascertain the relative positioning of a consonant sound, such as the consonant “b.” Consequently, these transcription technologies fail to discern, or to identify, in what position a patent has incorrectly or correctly pronounced the sound of the consonant “b,” whether in the initial position (“bass”), the middle position (“cabin”), and/or the final or end position (“shrub”) of a word).

Current technologies to reduce a provider's time spent on administrative tasks are limited to simple speech-to-text transcription, as previously discussed. Even technologies that offer summarization of the speech-to-text transcription into a provider note, however, do not cover the full range of post-appointment analysis tasks required of a provider in providing therapeutic services. While electronic medical records (“EMR”) enable more efficient recordkeeping than the previous practice of using pen and paper charting, on average it can take a provider between 5 and 10 minutes per patient to draft a full EMR visit note summarizing the session, especially in the common format of “SOAP” (subjective, objective, assessment, and plan). In a standard SOAP note, the provider lists a subjective summary of information received from the client, an objective summary of the provider's review of the patient during the session, an assessment of the patient's condition and progress, and a plan for the patient's continued treatment. For the assessment, a provider can be limited in collecting the data necessary to fully, quantitatively assess a patient's performance while also trying to provide the therapeutic services at the same time. For example, in speech therapy, a provider may miss or miscount the instances that a patient correctly pronounces a sound while they are planning or thinking of the next activity for the patient to do, for an efficient use of time within a session. Equally, a provider may also fail to observe circumstances in which a sound is correctly pronounced in one position of the word (e.g., the middle position), but is otherwise incorrectly pronounced in another position (e.g., the initial or end position).

In addition to the time spent drafting a SOAP note, providers can spend an additional 5-10 minutes per patient analyzing a patient's performance to complete progress tracking, where the provider compares the patient's performance during the appointment or session against the objectives or goals set forth in a plan of care. Most progress tracking is quantitative in nature, to ensure consistency throughout sessions during the course of treatment, especially when multiple providers are providing treatment or care-which is common in settings such as speech therapy, physical therapy, and occupational therapy in healthcare settings. Many patients can have multiple objectives or goals, both short-term and long-term, which requires numerous calculations of progress across various objectives or goals, repeated across each session. Tracking a patient's progress against their objectives or goals can be an important and useful tool to determine which patients have made sufficient progress to discontinue therapy, whether in totality or for a specific measure of care, and which patients may require more therapy to meet their objectives or goals of their plan of care.

In addition, providers may have to complete additional time-consuming tasks, such as drafting a layman's summary of their visit notes for use in a client portal or patient education, as well as planning for future sessions for that patient. Planning for future sessions can often include the provider conducting research to create activities that are appropriate for the patient's skill levels while targeting their specific needs and corresponding objectives within their plan of care. For pediatric patients especially, providers may have to take additional time to find and plan fun and engaging therapeutic activities, so that young patients are eager to engage in sessions and appointments to progress within their plan of care.

While software or other computer-implemented methods might exist that can enable a provider to dictate their notes, or even record patient sessions and appointments to generate a corresponding visit note, such technology does not analyze the content of the appointments or sessions to generate reports on patient performance within the appointment or session, or over time. Using technology to facilitate these tasks would not only represent an improvement in the technology, but also allow providers to see a greater volume of patients with the time saved due to technology. Such technology can also ensure that EMR notes are consistent across time and providers, and are objectively based on the contents of the appointment or session, improving the ability to truly assess patient performance within a session and across sessions. Implementing technology in assessing a patient and planning for the patient's future sessions can ensure that the patient is at a strategic advantage to meet their specific treatment objectives.

### BRIEF SUMMARY

The present disclosure addresses the problems identified above, amongst others. Implementations consistent with the present disclosure provide systems, apparatuses, and methods for evaluating word structures in audio recordings of patient speech are disclosed. The systems, apparatuses, and methods include a computer-implemented method for evaluating word structures in audio recordings of patient speech, for example, in speech therapy (both articulation and non-articulation), occupational therapy, physical therapy, and other medical contexts. The method can include establishing or updating a data module for a patient associated with a respective appointment, based at least in part on input data provided by a provider via a platform, recording or uploading a recording of an audio file including patient speech of the appointment, processing the audio file, analyzing the audio file, drafting post-appointment documentation based at least in part on an analysis of the audio file, tracking a progression of the patient for at least one objective within a plan of care across at least two appointments in time, and creating recommendations for a subsequent appointment, based at least in part on the analysis of the audio file and the plan of care for the patient. The progression tracking of the patient can be based at least in part on the input data and an analysis of the audio file. Input data may include at least one of a patient profile, a plan of care for the patient, at least one objective related to the plan of care for the patient, a configurable threshold related to each objective and whether it was met, at least one interest of the patient, a history for the patient (medical conditions, diagnoses, and treatments), an audio sample of the patient speaking, and an audio sample of the provider speaking.

Processing the audio file may further include converting the audio file into a different file format, transcribing words spoken by the patient and provider in the audio file, and diarizing the transcript. Transcription of the spoken words may be conducted at the phoneme level for each word. Analyzing the audio file may further include identifying at least one word from the transcript to be analyzed, which can correspond to the plan of care associated with the patient; isolating fragments of the audio file into sound clips corresponding to the words from the transcript; and evaluating the fragments to determine whether the objective of the plan of care for the patient was met during the appointment, and to what extent, which may be done using a configurable threshold. Where the plan of care for the patient does not include specified objectives, the system may generate an objective targeted during the appointment based on the contents of the transcript. The system may also redact any protected health information from the transcript or input data corresponding to the patient.

Drafting post-appointment documentation may further include generating a provider visit note corresponding to the appointment, editing the provider visit note as needed, and summarizing the provider visit note for the patient or their family. Tracking a progression of the patient within the patient's plan of care may further include extracting performance information from the analysis of the audio file and post-appointment documentation corresponding to the appointment (related to at least one objective for the patient to determine whether, and to what extent, the at least one objective may have been met during the appointment), comparing the extracted performance information from the appointment to similar extracted performance information from previous appointment(s) to determine whether and to what extent that at least one objective was met across at least two appointments in time, and summarizing the determination of whether and to what extent the at least one objective was met by the patient across at least two appointments in time (either visually or in a report). Creating recommendations for subsequent appointments, based on the analysis of the audio file and the patient's plan of care, may also include drafting a treatment plan for subsequent appointments for the patient based at least in part on input data corresponding to the patient profile (such as patient interests), and searching a resource library for recommended resources, references, or activities for the subsequent appointment. Such resources, references, or activities may be adapted or integrated into the treatment plan, which may include generating or updating the plan of care with new or revised objectives to aid the patient in progressing towards achieving their plan of care.

A system which may execute the computer-implemented method may comprise components such as a network, at least one electronic device configured to communicate with at least one server via the network, and at least one server. In addition, the system may also include a database. The server may have a storage medium having instructions stored thereon, with a processor configured to execute one or more instructions from the storage medium, and a communications unit to communicate with the system via the network. The processor can be configured to execute the method set forth above.

In the context of systems, apparatuses, and methods for evaluating word structures in audio recordings, a method for evaluating word structures in patient speech is provided herein. The method may commence with an operation of recording speech originating from one or more speakers to create an audio file. The audio file may have a native file format associated therewith. The method may continue with an operation of transcribing the audio file to generate a transcript that identifies one or more words in the speech originating from the one or more speakers. The method may continue with an operation of detecting one or more phonemes in the one or more words in the speech originating from the one or more speakers. Each of the one or more phonemes may correspond to a sound-based unit in a position in each of the one or more words. The method may continue by isolating the audio file into one or more audio fragments that correspond to the one or more phonemes in the one or more words. The method may continue by analyzing the one or more audio fragments to determine whether the one or more phonemes in the one or more words was pronounced correctly or incorrectly in the corresponding sound-based unit in the position in the one or more words. The method may further continue by scoring performance of the speech originating from the one or more speakers by calculating correct and incorrect pronunciations of the one or more phonemes in each of the one or more words. And, the method may yet further continue by extracting a success rate for the correct pronunciations as compared to the incorrect pronunciations for the one or more phonemes as with respect to the corresponding sound-based unit in the position in the one or more words to generate an objective targeting the one or more phonemes for subsequent performance of the speech originating from the one or more speakers for the purpose of tracking said subsequent performance.

In one exemplary aspect according to the above-referenced embodiment, the audio file has speech from more than one of the one or more speakers.

In another exemplary aspect according to the above-referenced embodiments, the method continues with an operation of diarizing the speech in the audio file to separately identify on the transcript which of the one or more speakers originated the one or more words in the speech.

In another exemplary aspect according to the above-referenced embodiments, the transcript has one or more timestamps associated therewith. Each of the one or more timestamps corresponds to each of the one or more words in the speech originating from the one or more speakers.

In another exemplary aspect according to the above-referenced embodiments, the position of the sound-based unit in each of the one or more words constitutes an initial, a middle position, or a final position of the one or more phonemes.

In another exemplary aspect according to the above-referenced embodiments, the native file format associated with the audio file is any one of .ogg, .mp3, .m4a, .wav, .mp4, .avi, .mov, and .m4v.

In another exemplary aspect according to the above-referenced embodiments, the objective for subsequent performance of the speech originating from the one or more speakers constitutes a configurable threshold for correct pronunciations for the one or more phonemes as with respect to the corresponding sound-based unit in a position in the one or more words.

In another particular embodiment of systems, apparatuses, and methods for evaluating word structures in audio recordings, a system for evaluating word structures in patient speech is provided herein. The system may include an electronic device, a server, and a network. The electronic device may be associated with one or more speakers, and the electronic device may have an audio-recording device that is connected thereto. The electronic device may a communications unit that allows for communicative coupling to the network. The server may have a communications unit for communication with the electronic device via the network. The server may have a processor configured to execute instructions residing on a storage medium. The server may be configured to create an audio file based upon speech recorded by the audio-recording device connected to the electronic device. The audio file may have a native file format associated therewith. The server may further be configured to transcribe the audio file to generate a transcript that identifies one or more words in the speech originating from one or more speakers. The server may further be configured to detect one or more phonemes in the one or more words in the speech originating from the one or more speakers. The one or more phonemes may correspond to a sound-based unit in a position in the one or more words. The server may further be configured to isolate the audio file into one or more audio fragments that correspond to the one or more phonemes in the one or more words. The server may further be configured to analyze the one or more audio fragments to determine whether the one or more phonemes in the one or more words was pronounced correctly or incorrectly by the one or more speakers in the corresponding sound-based unit in the position in the one or more words. The server may yet further be configured to score performance of the speech originating from the one or more speakers by calculating correct and incorrect pronunciations of the one or more phonemes in the one or more words. And, the server may yet further be configured to extract a success rate for the correct pronunciations as compared to the incorrect pronunciations for the one or more phonemes as with respect to the corresponding sound-based unit in the position in the one or more words to generate an objective targeting the one or more phonemes for subsequent performance of the speech originating from the one or more speakers for the purpose of tracking said subsequent performance.

In one exemplary aspect according to the above-referenced embodiment, the audio file has speech from more than one of the one or more speakers. And, the server is yet further configured to diarize the speech in the audio file to separately identify on the transcript which of the one or more speakers originated the one or more words in the speech.

In another exemplary aspect according to the above-referenced embodiments, the transcript has one or more timestamps associated therewith. Each of the one or more timestamps corresponds to each of the one or more words in the speech originating from the one or more speakers.

In another exemplary aspect according to the above-referenced embodiments, the objective for subsequent performance of the speech originating from the one or more speakers constitutes a configurable threshold for correct pronunciations for the one more phonemes as with respect to the corresponding sound-based unit in the position in the one or more words.

In yet another particular embodiment of systems, apparatuses, and methods for evaluating word structures in audio recordings, another method for evaluating word structures in patient speech is provided herein. The method may commence with an operation of inputting data corresponding to a profile of a speaker. Such data may include at least a configurable threshold for correct and incorrect pronunciations of one or more phonemes. The method may continue with an operation of recording speech from the speaker to create an audio file. The audio file may have a native file format associated therewith. The method may continue with an operation of generating a transcript that identifies one or more words in the speech from the speaker. The transcript may bear timestamps corresponding to each of the one or more words. The method may continue with an operation of dissecting the audio file into one or more audio fragments to target one or more phonemes in the one or more words. The one or more phonemes may correspond to a sound-based unit in a position in the one or more words. The method may continue with an operation of determining whether the one or more phonemes in the one or more words was pronounced correctly or incorrectly by the speaker in the corresponding sound-based unit in the position in each of the one or more words. The method may continue with an operation of scoring performance of the speech of the speaker by calculating correct and incorrect pronunciations of the one or more phonemes in the one or more words. The method may further continue with an operation of performing error analysis of the performance of the speech by comparing the correct and incorrect pronunciations of the one or more phonemes in the one or more words against the configurable threshold to determine a success rate for the correct pronunciations for the one more phonemes as with respect to the sound-based unit in the position in the one or more words. And, the method may yet further continue with an operation of creating an objective for subsequent performance of the speech based upon the error analysis. The objective for subsequent performance may prescribe an updated configurable threshold for correct and incorrect pronunciations for the one more phonemes as with respect to the sound-based unit in the position.

In one exemplary aspect according to the above-referenced embodiment, the audio file has speech from one more speakers other than the speaker.

In another exemplary aspect according to the above-referenced embodiments, the method continues with an operation of diarizing the speech of the speaker from the speech of the one or more speakers in the audio file by separately identifying on the transcript which of one or more speakers or the speaker originated the one or more words in the speech, and comparing the one or more words in the speech from the one or more speakers and the speaker with a sampled audio recording of speech attributable to the speaker. The sampled audio recording is included in the data corresponding to the profile of the speaker.

In another exemplary aspect according to the above-referenced embodiments, the operation of creating the objective for subsequent performance of the speech based upon the error analysis includes comparing the one or more words of the transcript against a bank of resources comprising word-embedded metadata.

In another exemplary aspect according to the above-referenced embodiments, comparing the one or more words of the transcript against a bank of resources comprising word-embedded metadata includes performing a word vector search of the word-embedded metadata to identify other one or more words having the one or more phonemes pronounced incorrectly by the speaker.

In another exemplary aspect according to the above-referenced embodiments, the operation of creating the objective for subsequent performance of the speech based upon the error analysis includes analyzing correct pronunciations for the one or more phonemes in the one more words in a corresponding first sound-based position against incorrect pronunciations for the one or more phonemes in the one or more words in a corresponding second sound-based position.

In another exemplary aspect according to the above-referenced embodiments, the operation of scoring performance of the speech by calculating correct and incorrect pronunciations of the one or more phonemes in the one or more words depends upon the pronunciation of the corresponding sound-based unit in the position in the one or more words.

In another exemplary aspect according to the above-referenced embodiments, the position of the sound-based unit in the one or more words constitutes an initial position, a middle position, or a final position of the one or more phonemes.

In another exemplary aspect according to the above-referenced embodiments, the operation of scoring performance of the speech by calculating correct and incorrect pronunciations of the one or more phonemes in each of the one or more words is limited to a single sound-based unit in a single position in each of the one or more words. The single position of the single sound-based unit is one of the initial, middle, or final position of the one or more phonemes.

Other objects and advantages of this disclosure will become readily apparent from the ensuing description.

Unless otherwise defined, all technical and scientific terms used herein have the same meaning as commonly understood by one of ordinary skill in the art to which this disclosure belongs. The present disclosure may be embodied in other specific forms without departing from the spirit or essential attributes thereof, and it is therefore desired that the embodiments of the disclosure be considered in all aspects as illustrative and not restrictive. Any headings utilized in the description are for convenience only and no legal or limiting effect. Numerous objects, features, and advantages of the embodiments set forth herein will be readily apparent to those skilled in the art upon reading of the following disclosure when taken in conjunction with the accompanying drawings.

### BRIEF DESCRIPTION OF THE DRAWINGS

Certain illustrations, charts, or flow charts are provided to allow for a better understanding of the present disclosure. It is to be noted, however, that the drawings illustrate only selected exemplary embodiments of the disclosure and are therefore not to be considered limiting of scope. Additional and equally effective embodiments and applications of the present disclosure exist. Hereinafter, various exemplary embodiments of the disclosure are illustrated in more detail with reference to the drawings.

FIG. 1 is an exemplary embodiment of a system for evaluating word structures in audio recordings of patient speech, in accordance with aspects of the present disclosure.

FIG. 2 is an exemplary embodiment of a partial block diagram of a database, in accordance with aspects of the present disclosure.

FIG. 3 is an exemplary embodiment of a partial block diagram of a server, in accordance with aspects of the present disclosure.

FIG. 4 is an exemplary embodiment of a partial block diagram of an electronic device, in accordance with aspects of the present disclosure.

FIG. 5A is a flowchart illustrating an exemplary embodiment of a method of evaluating word structures in audio recordings of patient speech, in accordance with aspects of the present disclosure.

FIG. 5B is an exemplary embodiment of a method to process an audio-file recording, implementable in conjunction with the method of FIG. 5A and in accordance with aspects of the present disclosure.

FIG. 5C is an exemplary embodiment of a method to analyze an audio-file recording, implementable in conjunction with the method of FIG. 5A and in accordance with aspects of the present disclosure.

FIG. 5D is an exemplary embodiment of a method to draft post-appointment documentation based on an audio-file recording, implementable in conjunction with the method of FIG. 5A and in accordance with aspects of the present disclosure.

FIG. 5E is an exemplary embodiment of a method to track a progression of a patient across appointments based on an audio-file recording, implementable in conjunction with the method of FIG. 5A and in accordance with aspects of the present disclosure.

FIG. 5F is an exemplary embodiment of a method to create recommendations for a subsequent appointment(s) based on an audio-file recording, implementable in conjunction with the method of FIG. 5A and in accordance with aspects of the present disclosure.

FIG. 6 is an exemplary embodiment of an excerpt of performance in an evaluation of word structures in audio recordings of patient speech, in accordance with aspects of the present disclosure.

### DETAILED DESCRIPTION

Detailed descriptions of one or more embodiments are provided herein. It is to be understood, however, that the present disclosure can be embodied in various forms. Therefore, specific details disclosed herein are not to be interpreted as limiting, but rather as a basis for the claims and as a representative basis for teaching one skilled in the art to employ the present disclosure in any appropriate manner.

The singular forms “a,” “an,” and “the” include plural reference unless the context clearly dictates otherwise. The use of the word “a” or “an” when used in conjunction with the term “comprising” in the claims or the specification can mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The terms “comprising” and “including” and “having” and “involving” (and similarly “comprises,” “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b, and c. Wherever the terms “a” or “an” are used, “one or more” is understood, unless such interpretation is nonsensical in context.

The terms “individual,” “patient,” “student,” “and “client” refer to an entity, e.g., a human, participating in an appointment or session that uses a system, apparatus, or method evaluating word structures in audio recordings of patient speech as disclosed herein. And, in certain contexts, the term “speaker” refers to an entity, e.g., a human, that engages with, participates in, or uses the system, apparatus, or method evaluating word structures in audio recordings of patient speech as disclosed herein; it being understood that the “speaker” can encompass the individual (or individuals) from which the speech is originating or being spoken. These terms as used herein refer to one or more individuals, patients, students, clients, or speakers.

The term “provider” refers to an entity, e.g., a human, using, engaging with, participating in, or directing a system, apparatus, or method evaluating word structures in audio recordings of patient speech as disclosed herein. The term “provider” herein refers to one or more providers.

The term “platform” refers to a hosted platform related to software that is executing on an electronic device such as a smartphone, tablet, and/or web browser on any computing device, whether as a downloadable instance locally or on premise, or accessible or useable as under a software-as-a-service (SaaS) model.

The terms “connection” or “connected” refers to connecting any component as defined below by any means, including but not limited to, a wired connection(s) using any type of wire or cable for example, including but not limited to, coaxial cable(s), fiberoptic cable(s), and ethernet cable(s) or to wireless connection(s) using any type of frequency/frequencies or radio wave(s). Some examples are including below in this application.

The below detailed description is provided for the purposes of illustration and description. Thus, although there have been described particular embodiments of new and useful SYSTEMS, APPARATUSES, AND METHODS FOR EVALUATING WORD STRUCTURES IN AUDIO RECORDINGS OF PATIENT SPEECH, it is not intended that such references be construed as limitations upon the scope of this disclosure except as set forth in the appended claims. Thus, it is seen that methods and systems of the present disclosure readily achieve the ends and advantages mentioned as well as those inherent therein. While certain preferred embodiments of the disclosure have been illustrated and described for present purposes, numerous changes in the arrangement and construction of parts and steps may be made by those skilled in the art, which changes are encompassed within the scope and spirit of the present disclosure as defined by the appended claims.

Reference will now be made in detail to embodiments of the present disclosure, one or more drawings of which are set forth herein. Each drawing is provided by way of explanation of the present disclosure and is not a limitation. In fact, it will be apparent to those skilled in the art that various modifications and variations can be made to the teachings of the present disclosure without departing from the scope of the disclosure. For instance, features illustrated or described as part of one embodiment can be used with another embodiment to yield a still further embodiment.

While the making and using of various embodiments of the present disclosure are discussed in detail below, it should be appreciated that the present disclosure provides many applicable inventive concepts that can be embodied in a wide variety of specific contexts. The specific embodiments discussed herein are merely illustrative of specific ways to make and use the technology and do not delimit the scope of the disclosure.

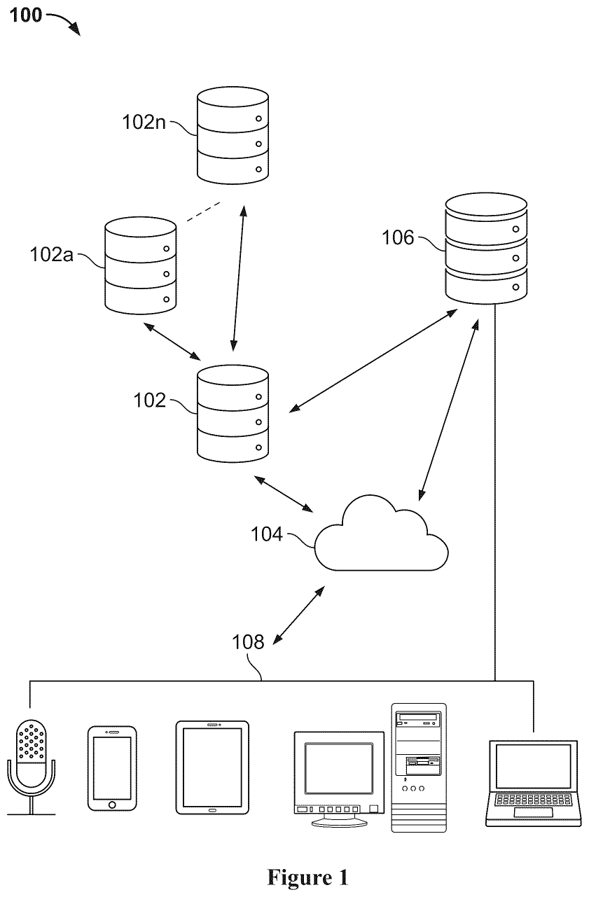

Thus, it is intended that the present disclosure covers such modifications and variations as come within the scope of the appended claims and their equivalents. Other objects, features, and aspects of the present disclosure are disclosed in, or are obvious from, the following detailed description. It is to be understood by one of ordinary skill in the art that the present discussion is a description of exemplary embodiments only and is not intended as limiting the broader aspects of the present disclosure. Referring generally to FIGS. 1-6, various exemplary embodiments the present disclosure may now be described in detail, including a system 100 and method 500 for evaluating word structures in audio recordings of patient speech. Where the various figures may describe embodiments sharing various common elements and features with other embodiments, similar elements and features are given the same reference numerals and redundant description thereof may be omitted below.

Referring to FIG. 1, provided is an embodiment of the system 100, that can evaluate word structures in audio recordings of patient speech. The system 100 can provide use of online, non-downloadable cloud computing software enabling users such as providers or teachers to record, process, and analyze appointments or sessions with patients or students. In other embodiments, the software may be downloadable to electronic devices equipped with a network connection. The system 100 may comprise one or more server(s) 102, 102a . . . , 102n, a network 104, a database 106, and at least one of a set of supported electronic devices 108 associated with a user (including a voice recorder, smartphone, a tablet, a personal computer, a laptop, and other mobile or computing devices (not shown)). Those skilled in the art with reference to this disclosure should appreciate that other configurations may be used to accomplish the methods described herein without departing from the scope of the present disclosure. For purposes of this disclosure, reference to a server, database, or processor, shall be interpreted to include: a single server, a single processor, a single database, multiple servers, multiple processors, multiple databases, or any combination of server(s), processor(s), and database(s).

The server 102 may be configured to store, access, or provide at least a portion of information usable to permit one or more operations described herein. For example, the server 102 may be configured to provide a portal, webpage, interface, and/or non-downloadable application to an electronic device 108 to enable one or more operations described herein. The server 102 may additionally or alternatively be configured to store content data and/or metadata to enable one or more operations described herein.

As shown in FIG. 1, in one embodiment, the server 102 may control one or more operations of the system 100, as discussed herein. The electronic device 108 can be connected to the server 102 via the network 104. In an embodiment, the operation of the system 100 is implemented electronically by software that runs on or is otherwise associated with the server 102. The electronic devices 108 can connect to the network 104 to communicate with the server 102 or database 106. Components of the system 100 that interact with the network 104 (server 102, database 106, and electronic device 108) can have a communications unit 206, 306, 406 that enable the components to communicate with the network 104 or other components of the system 100.

The server 104 may be configured to communicate data to and from various devices in the system (including other servers 102a-102n, the database 106, or the electronic device 108) and to perform one or more method steps, as detailed below. The server 102 may contain various types of data and computer instructions for performing at least some of the steps presented herein. There may be other servers 102a-102n that can interact with the server 102, or other components of the system 100, via the network 104.

The database 106 may store various types of data or instructions for performing at least some of the steps and operations presented herein, including the operations prescribed or otherwise set forth in the method 500 for evaluating word structures in audio recordings of patient speech. In embodiments where there is only one server 102 (as shown in FIG. 1) without a database 106 in the system 100, where any of the disclosure below includes a description of a database 106 or what steps the database 106 conducts within the system 100, all such description, steps, and actions can instead either describe or be conducted by the server 102, or by multiple servers 102a-102n.

The network 104 need not be a single network (such as only the internet) and may be multiple networks (whether connected to each other or not). It should be understood that FIG. 1 is an exemplary embodiment of the present system and various other configurations are within the scope of the present system. For example, one or more of the electronic devices 108 and server 102 may all be located in different locations, where all of these components of the system 100 are operatively coupled by a network 104. Similarly, the server 104 may be local to the database 106 or located in a different location. The network 104 may be comprised of multiple servers 102a-102n, and multiple databases 106, and one or more electronic devices 18, whether located locally and networked through a LAN or remotely through a WAN or an Intranet connection. As shown in FIG. 1, in a multiple-server configuration, the server 102 can facilitate communications between the other servers 102a-102n; in other embodiments of the system 100, the other servers 102a-102n may directly communicate with the other components of the system 100 using the network 104.

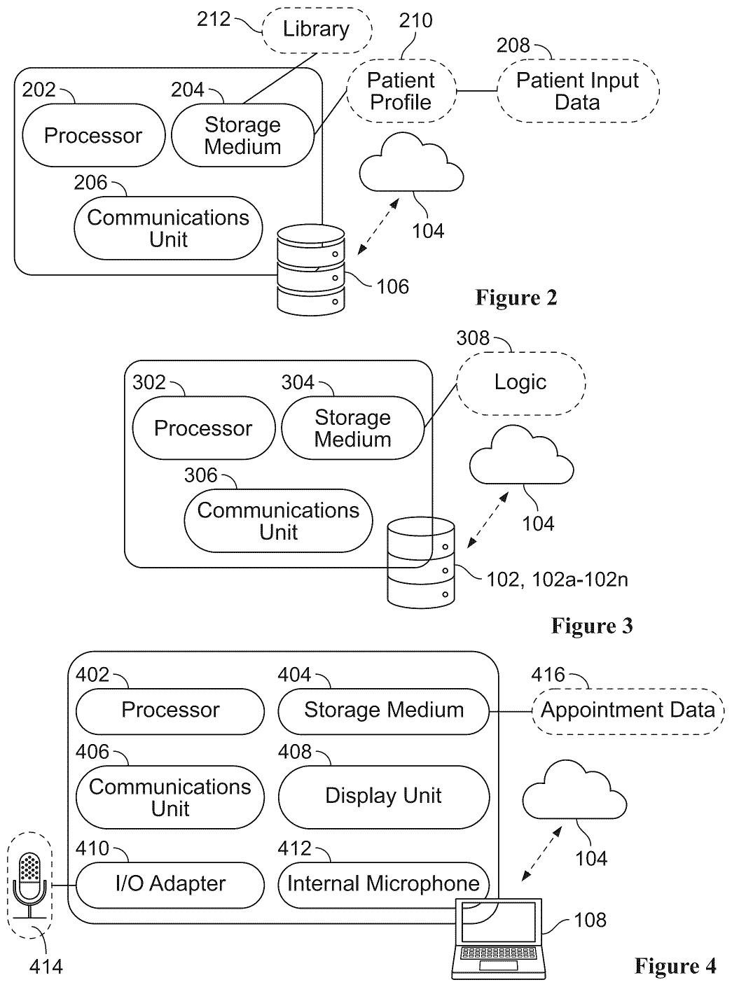

FIG. 2 illustrates an exemplary embodiment of a partial block diagram of the database 106. The database 106 may include at least one processor 202, storage medium 204, and communications unit 206. The database 106 may have other software components, such as a database engine (not shown), such as those allowing for security mechanisms to protect data stored on the storage medium 204 (including authentication, authorization, encryption, redaction, anonymization, de-identification, pseudonymization, and auditing features), backup and recovery mechanisms, and more.

Similarly, FIG. 3 illustrates an exemplary embodiment of a partial block diagram of the server 102, 102a-102n. The system 100 may have only one server 102 or may have multiple servers 102, 102a-102n. Each server 102, 102a-102n, may include at least one processor 302, storage medium 304, and communications unit 306. For embodiments of the system 100 that have multiple servers 102, 102a-102n, each server 102, 102a-102n may handle different steps of the below-mentioned method 500 and some other servers 102a-102n may be external to the system 100; for example, one server 102a may handle operations 510-520b, 550-570, while another server 102n may handle operation 530, and yet another server 102n may handle operation 540 (or any other combination of servers and operations thereof).

Where any of the disclosure above or below includes a basic description of a particular component of, or the functionality of, the database 106 (such as the processor 202, 302, the storage medium 204, 304, or the communications unit 206, 306), all such descriptions or functions can either describe or be conducted by the companion component in the server(s) 102, 102a-102n. For example, the communications unit 306 of the server 102 can have the same structure and function as the communications unit 206 of the database 106.

FIG. 4 illustrates an exemplary embodiment of a partial block diagram of the electronic device 108. The electronic device 108 can be any one or more of a voice recorder, smartphone, a tablet, a personal computer, a laptop, or other mobile or computing device. The electronic device 108 may include a processor 402, a storage medium 404, a communications unit 406, a display unit 408, an input/output (I/O) adapter 410 that can communicate with an external audio-recording device 414 or microphone device 414, particularly in embodiments in which the electronic device 108 does not have an internal microphone 412, a user interface adapter (not shown) configured to link a user input device(s) (such as a mouse, keyboard, touch-screen interface, and the like) (not shown) to the electronic device 108, and the internal microphone 412. Again, if the electronic device 108 is not equipped with an internal microphone 412 or other audio-recording device 412, the external microphone 414 or other audio-recording device 414 may be used with the electronic device 108 such that it can record audio. In some embodiments of the system 100, there may be more than one external microphone 414, which may or may not be used in conjunction with the internal microphone 412, depending on the number of individuals speaking during an appointment or a session (patient, provider(s), parent(s), student-provider(s), and the like), such that each speaker may have their own microphone 412, 414. In other embodiments of the system 100, the electronic device 108 used by a provider may receive a copy or upload of a file with an audio recording via the network 104 from either another electronic device 108 or the database 106. In still other embodiments of the system 100, the server 102 may directly receive a copy or upload of a file with an audio recording via the network 104 from either an electronic device 108 or the database 106. The electronic device 108 may also have a network connection card within its communications unit 406 to connect to the system 100 via the network 104.

The processor 202, 302, 402 may be a high-performance processor, a generic hardware processor, a special-purpose hardware processor, or a combination thereof. In embodiments having a generic hardware processor (e.g., as a central processing unit (CPU) available from manufacturers such as Intel and AMD), the generic hardware processor may be configured to be converted to a special-purpose processor by means of being programmed to execute and/or by executing a particular algorithm in the manner discussed herein for providing a specific operation or result. It should be appreciated that the processor 202, 302, 402 may be any type of hardware and/or software processor and is not strictly limited to a microprocessor or any operation(s) only capable of execution by a microprocessor, in whole or in part.

The storage medium 204, 304, 404 may include various memory devices, which may include random access memory (RAM), which may be synchronous RAM (SRAM), dynamic RAM (DRAM), synchronous dynamic RAM (SDRAM), or the like, or read only memory (ROM) which may be PROM, EPROM, EEPROM, optical storage, or the like; the server 102, database 106, or electronic device(s) 108 may utilize the RAM to store the various data structures used by a software application to operate the system 100 and the ROM to store configuration information for booting the system 100. The RAM and the ROM are memory devices that can hold user and system data, and both the RAM and the ROM may be randomly accessed. Data storage using the storage medium 204, 304, 406 may include a separate server 102a-102n coupled to the server 102, or system 100 through the network 104. If the storage medium 204, 304, 406 is not a physical memory component within the server 102, the database 106, or the electronic device 108, the storage medium 204, 304, 406 can be an external memory component (not shown) coupled to the I/O Adapter 410, or a cloud-based memory system accessible to the server 102, the database 106, and/or the electronic device 108 via the network 104.

The processor 202 of the database 106 can dynamically and efficiently pull data, such as the patient input data 208, from the storage medium 204 of the database 106 for use within the system 100, via the communications unit 206. For example, the patient voice sample stored in the patient input data 208 from the storage medium 204 of the database 106 can be communicated to the system 100 via the communications unit 206 of the database 106 for use by the processor 302 of the server 102. For example, the processor 302 can analyze the voices within a recording of an appointment or session to separate out different speakers, determine which speaker's voice matches the patient voice sample stored in the patient input data 208, and then label that speaker as the patient throughout a transcript generated from the recording of the appointment or session.

For embodiments of the system 100 without a database 106, the above functions are served by the processor 302 and storage medium 304 of the server 102. In embodiments of the system 100 with multiple servers 102a-102n, the processor 302 can cause the communications unit 306 of one server 102a to transmit data stored on the storage medium 306 of one server 102a to another server 102n, for use by the processor 302 of the other server 102n.

The processor 302 of the server 102 can retrieve and execute the instructions in the logic 308 (further described below) from the storage medium 304 of the server 102 to execute various functions of the system 100. Specifically, the storage medium 304 of the server 102 may store logic 308 such as information and instructions for the processor 302 to carry out the operations disclosed herein, including the operations as prescribed and set forth in the method 500.

Non-limiting examples of the information stored on the storage medium 304 include information and instructions on how to retrieve information from the memory or storage medium(s), enable the smooth data flow to various components of the system 100, how to manage various information or data used by the system 100 (including, but not limited to, patient input data 208 and appointment data 416, logic 308 for data transfers between various components of the system 100, logic 308 to trigger an information exchange between various components of the system 100, how to process data or information received from other system components (including, but not limited to, a server 102, a database, 106, an electronic device 108) via the network 104, how to direct an electronic device 108 to record audio, how to process an audio file, how to convert various types of audio files, how to create a transcript corresponding to the audio file, how to redact protected health information (“PHI”) from a transcript or patient input data, how to diarize speakers within the audio file, how to analyze the audio file, how to identify significant words within an audio file, how to isolate audio fragments of the audio file corresponding to significant words, how to conduct a pronunciation analysis of the significant-word fragments (including scoring to create objective data), how to conduct other analyses of significant-word fragments (including scoring to create objective data to determine whether an objective of a plan of care was met for the patient and to what extent), how to use data from the analysis and transcript to create an objective or goal for a plan of care for a patient, how to use data from the analysis to generate or draft a visit note, how to allow a provider-user to edit the generated visit note, how to use data from the analysis to track patient progress and represent said progress visually, how to track patient progress over time by comparing objective data across sessions, how to convert the transcript and visit note into a patient-friendly summary, how to apply word embedding to the transcript, how to use the transcript to conduct a word vector search of a library or list of references, resources, and activities to plan future sessions, how to adapt resources and activities from the library 212 for use with a specific patient and specific objective(s) of a plan of care for that patient, how to store various information (such as the objective data, other analyses results, transcripts, and visit notes) within the system 100 by associating said information with a patient profile 210, how to control other administrative or backend features explained herein, how to apply other policies or features of the system 100, and how to carry out or implement the operations of the method 500, and more.

The processor 402 of the electronic device 108 can record audio of the appointment or session using either an internal microphone or recording device 412 or external microphone(s) or recording device(s) 414.

In an embodiment of the system 100 with a database 106, the storage medium 204 can store patient input data 208; this function can be served by a storage medium 304 of a server 102 in embodiments of the system 100 without a database 106. In any embodiment of the system 100, and within any component of the system 100, the use, communication, and storage of such patient input data 208, including PHI within patient records, comply with HIPAA and state/federal data privacy legislation, as well as any written consent(s) required for the use of audio recording during appointments or sessions (and the subsequent use of that recording). Patient input data 208 can represent the data entered into the system 100, via an electronic device 108 by a provider or patient, including, but not limited to, at least one of the following: the full name of the patient, the age and date of birth of the patient, interests of the patient, a plan of care for the patient with short-term or long-term objectives or goals, and a sample of the patient's voice. The sampling of the patient's voice can be an audio recording of the patient reading a predetermined sentence such that the system 100 can learn and understand how the patient's voice sounds. Such audio may be recorded by the internal microphone 412 or the external recording device 414 of the electronic device 108. The patient input data 208 can be stored in the form of a patient profile 210 within the storage medium 204, 304 of the database 106 or server 102. The patient input data 208 or patient profile 210 may also be associated with a profile corresponding to the provider that is offering the therapeutic services; the provider profile (not shown) may include an audio recording file of the provider's voice (also stored in the storage medium 204, 304), such that the system 100 can learn and understand how the provider's voice sounds and differentiate the provider's voice from the patient's voice by comparing the voice samples of the provider and patient.

For servers 102 that can interact with another server 102a-102n via the network 104, the storage medium 304 of the server 102 may store instructions configured to be executed by the processor 302 of the server 102 to interact with another server 102a-102n, including how to interact with any application programming interface (“API”) of these servers 102a-102n. If the embodiment of the database 106 has an API, the server 102 can have the programming or application logic, stored in the storage medium 304 of the server 102, necessary to interact with the API of the database 106. The ability of the server 102 to integrate with the API of another component of the system 100 can help ensure a reliable and efficient data exchange to help the overall system 100 run smoothly by leveraging the capabilities of the other server 102a-102n. The system 100 can be integrated with other external systems (not shown); such integration may also use an API specific to the external system.

The communications unit 206, 306, 406 may be adapted to couple the server 102, database 106, or electronic device 108 to the network 104 (which may be one or more of a LAN, WAN, and/or the Internet which may be performed by wired interface, wireless interface, or a combination thereof) to communicate with other components of the system 100, other electronic devices 108, or other networks such as a global positioning system (GPS) or a Bluetooth network.

As shown in FIG. 4, the display unit 408 may interact with other components of the electronic device 108 to display content as a user interacts with the electronic device 108. The display unit 408 may be driven by the processor 402 to control the display, and may interact with other components of the electronic device 108, such as a display driver (not shown); the input/output (I/O) adapter 410 (including, for example, various circuity and processing components from incoming video signals such as HDMI, USB, or network connections via the network 108 or the external microphone or recording device 414); a user interface adapter (not shown) configured to link a user input device (not shown) (such as a keyboard, mouse, touch-display, or other user interface) to the electronic device 108; and a display panel (not shown) (which may be made of any kind of display panel known in the art, including, for example, LCD, LED, OLED, QLED, MicroLED, a light source, and the like). The electronic device 108 may include speakers or some other audio output component (not shown), the internal microphone or recording device 412 or the external microphone or recording device 414 input component.

The system 100 may utilize machine learning and artificial intelligence (“AI”). In such embodiments, audio files can be processed and analyzed using prompts to AI algorithms to achieve various functions. In such embodiments, another server 102a-102n may be dedicated to the hosting of an AI system, including systems practicing machine learning and deep learning, wherein said other server 102a-102n can interact with the system 100 using the communications unit 206 via the network 104; in such embodiments of this system 100, an AI model (not shown) is stored within the storage medium 304 of said server 102a-n and executed by the processor 302. The system 100 may use multiple such AI models. Specified prompts to generate the output utilized by the system 100 can be stored in the storage medium 304 of the server 102 and executed by the processor 302; in embodiments where the AI system is hosted by another server 102a-102n, the processor 302 of the server 102 may also have instructions on how to interact with the other server 102a-102n including via an API.

Such prompts can include various prompts to process and analyze an audio file, generate diarized transcripts and a visit note in the SOAP format, summarize a transcript and visit note for patients or their parents, use objective data from the analysis and transcript to track patient progress against objectives, generate said objectives where needed, and plan for future treatment sessions (including the searching and adaptation of resources, references, and activities from a library), and the like. The prompts may be detailed and specific to the area of therapy or medicine in which the system 100 is being deployed; the exact form of the prompts can alter the output from the AI system. For example, a prompt may upload a redacted audio file to the system to create a transcript, or supply objectives from a redacted plan of care to the AI system and ask the system to apply the objectives to a transcript to determine whether such objectives were met, tracking the patient's progress within those objectives, and utilizing resource or activity libraries to plan for future sessions to target those objectives. The AI system can be trained and prompted to avoid AI hallucinations, in which the system makes up information or data for use in its output; avoiding such hallucinations in the output of any AI systems may be preferable for the use of such output by the system 100 that creates content for a patient's EMR, which must be accurate, valid, and truthful to the patient's appointment.

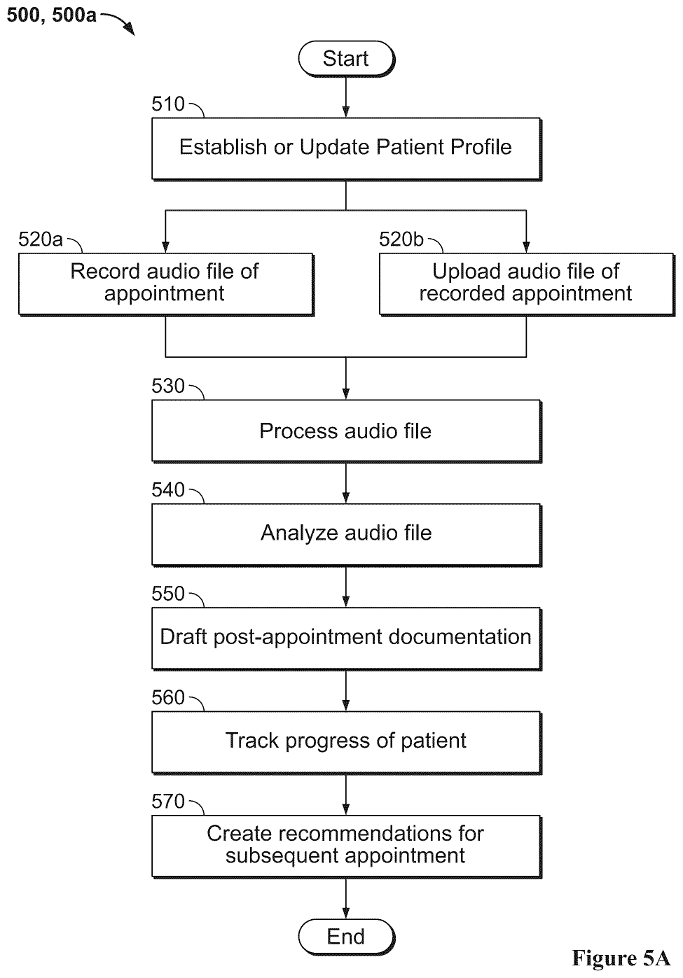

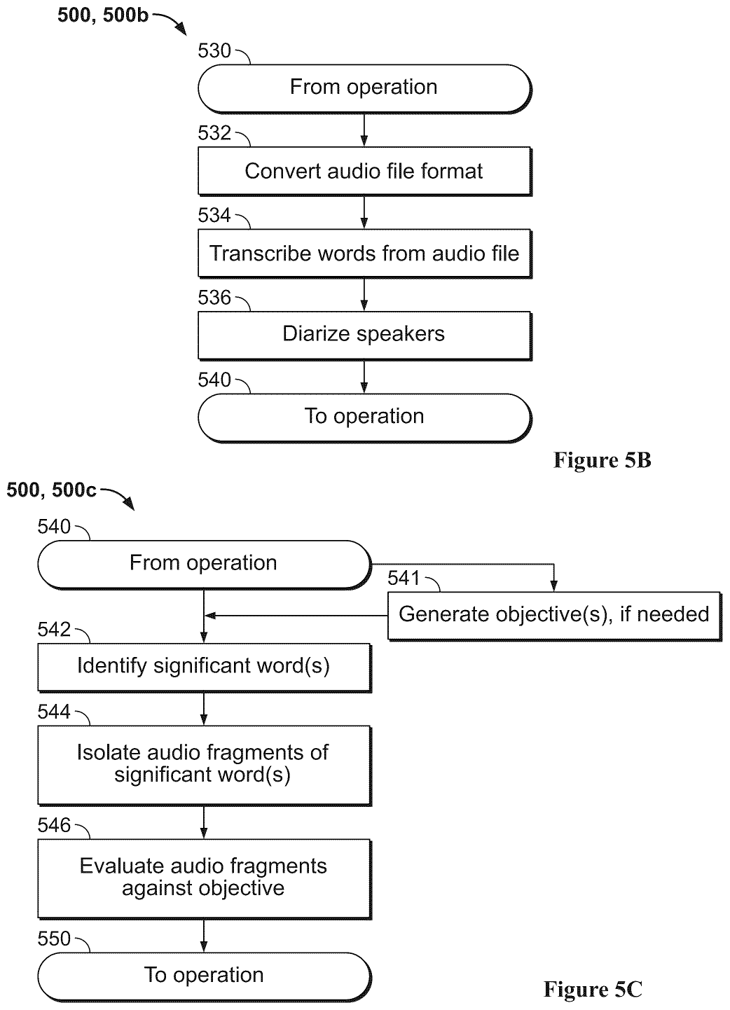

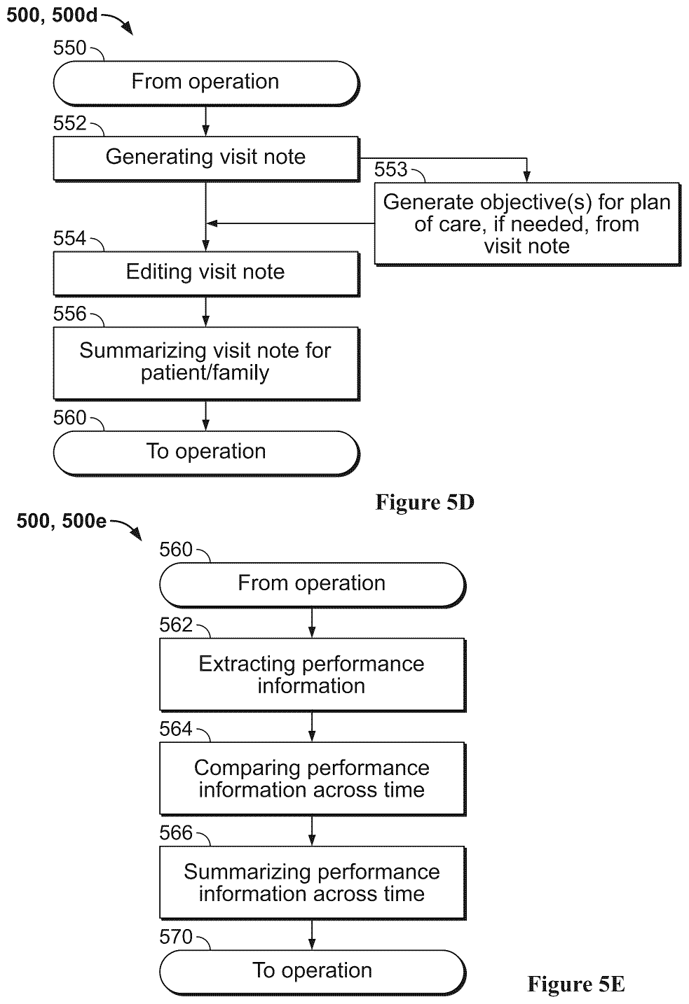

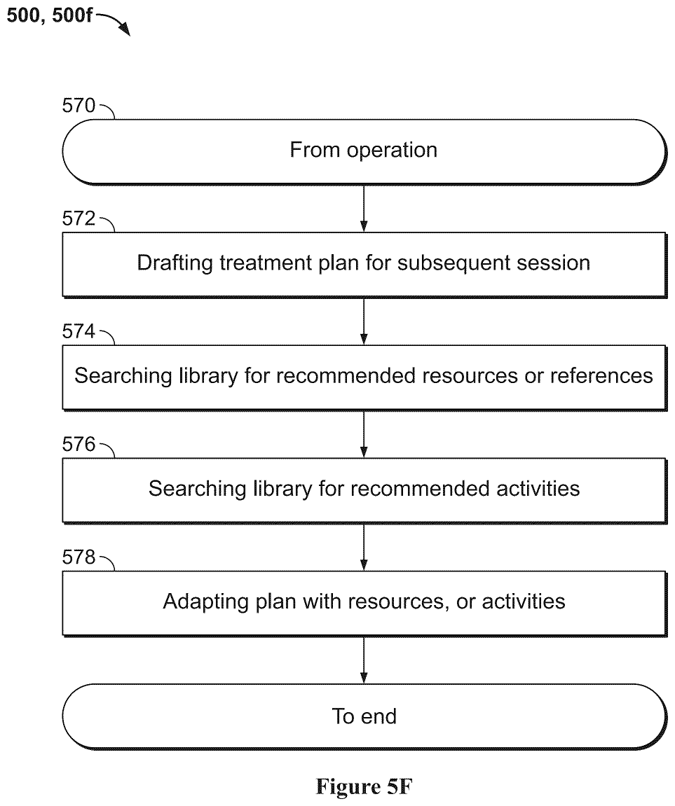

FIGS. 5A-5F illustrate flowcharts providing an exemplary embodiment of the computer-implemented method 500 of evaluating word structures in audio recordings of patient speech. Certain operations or steps of the method 500 can have operations 500a and sub-operations 500b-500f, as illustrated in FIGS. 5A-5F. As set forth below, the sub-operations 500b-500f, as illustrated in FIGS. 5B-5F, will be discussed in line with the overall method 500a, as illustrated in FIG. 5A, where appropriate.

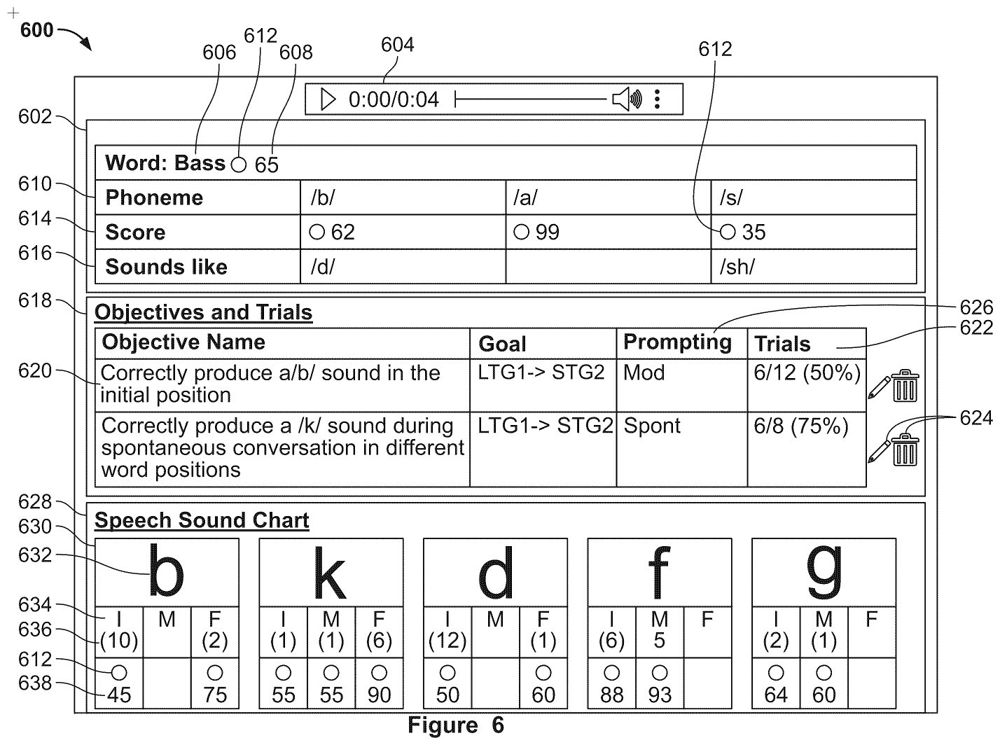

FIG. 6 is an exemplary embodiment of an excerpt of a report 600 of performance in an evaluation of word structures in audio recordings of patient speech associated with a therapy appointment produced by the method of FIGS. 5A-5F, in accordance with aspects of the present disclosure, and will be discussed in line with the overall method 500a, where appropriate.

The method 500a can begin by the operation 510 of establishing or updating a data module for a patient, or a patient profile 210, the patient being associated with a respective appointment. The patient profile 210 can be based at least in part on input data provided by a provider via a platform, the platform being hosted on a server 102. The patient profile 210 can also input data from the patient, a patient's parent (if a minor), or other third parties (such as a patient's teacher, or a referring third-party provider); such non-provider data input can be via intake forms (physical or electronic) or other similar documentation (such as school plans, such as IEP, 504, or other services plans-the system 100 may also alert providers as to when such time-sensitive documentation may expire). The processor 202 of the server 102 can receive such data input to create or update the patient profile 510 from an electronic device 108 coupled to the system 100 via the network 104.

The input data forming the patient profile 210 can include various PHI of the patient, such as their name, date of birth, age, insurer, referring physician, gender/sex, grade level, addresses, parent contact information (if a minor patient), and the like. Also included in a patient profile 210 can be a plan of care for the patient, which can be further broken down into at least one objective or goal, either short-term or long-term, for the patient to accomplish during the therapy appointments or sessions. The at least one objective or goal can include both a baseline level (which represents the patient's level of scored performance when the objective or goal was first tested) and a target level or configurable threshold (which represents a target/threshold for when the patient has met the objective or goal). For example, if the patient is undergoing speech therapy, an exemplar articulation objective or goal can be to correctly produce a/b/sound during spontaneous conversation in different word positions (i.e., an “b” sound in different word positions: the initial (“bass”), middle (“cabin”), and final or end (“shrub”) of a word); the configurable threshold can be set to 70 for this objective, such that the patient is considered to have met the objective or goal when 70% of their “b” sounds are correct during the appointment in different word positions, such that the goal is objectively and quantitatively measured as a function of achieved objective instances out of all objective instances (i.e., out of 20 instances where an “b” sound was in a word during the appointment, if the patient only correctly pronounced the “b” sound 13 instances, the patient achieved a score of 13/20 or 65%, lower than the 70% threshold and therefore did not meet the objective during this appointment). The plan of care can further include information input by the provider, such as prognosis, impression(s), and recommendation(s) for therapy.

As discussed in more depth below with FIG. 5C, the system 100 does not require the input data for a patient profile 210 to function, but the output of the system can be different when there is no such plan of care and related objectives or goals. A provider can also configure the settings of the system 100 based on the type of therapeutic services the provider renders. For example, in speech therapy, the therapist can enter a pronunciation score threshold for the patient, and enable or disable the error analysis of words at a phoneme level based on whether a patient is targeting articulation. The configurable threshold of the patient's objectives can be manually entered and altered based on level of the patient or the provider's preferences (for example, for a patient that is in the earlier stages of therapy, the provider may lower the threshold so it is easier for the patient to achieve a correct trial). The configurable threshold may represent a configurable scale instead; for example, instead of a simple pass/fail based on a threshold number, there can be a scoring scale (A through F, excellent through poor, etc.) Pronunciation error analysis may be disabled for therapy services outside of the speech therapy area, such as physical or occupational therapy or other medical applications of the system 100.

The input data for the patient profile 210, such as the patient input data 208, may also include at least one interest of the patient, which can be used by the system 100 to later adapt activities and resources for use for that patient. Patient interests can be especially important for younger, pediatric patients, as incorporating their interests into the therapy appointments or sessions can be more engaging for the patient, increasing the likelihood that the younger patient is willing to go to therapy and do the activities during therapy to learn the skills within their plan of care. The input data for the patient profile 210 may also include a history of the patient's medical history, previous and current conditions, diagnoses (with corresponding codes), and treatments for the patient. For a new patient, the patient profile 210 can be established; for an existing patient, the patient profile 210 can be updated (if there are no updates to the patient profile 210, this operation 510 can be skipped to proceed directly to operation 520a or 520b). For current patients, the plan of care and its related objective(s), the patient's interests, or other historical questions may require updating before each session or appointment.

The patient profile 210 can be linked to any saved audio recordings of sessions or appointments, saved sound clips from said audio recordings, visit notes, progress tracking, recommendations for future sessions, and the like. In this way, the patient profile 210 can function as an EMR for the patient. The patient profile 210, or at least some of the information stored in the patient profile 210, may be accessible to the patient (or for pediatric patients, their parent or guardian) using a patient portal system (not shown); this patient portal system can be hosted on the server 102, or another server 102a-102n on the system 100, and may include data that is stored in the storage medium 204, 304 of the database 106, server 102, or another server 102a-102n or external memory device (not shown). The patient profile 210 may allow for other features through the portal system, such as secure messaging between the patient and the provider (and in some cases a third party, for example, a pediatric patient's teacher), the ability to send alerts to the patient, the ability for both patients and providers to upload documents to the system 100 in various file formats (including, but not limited to, .doc, .docx, .ppt, .pptx, .csv, .xls, .xlsx, .pdf, .gif, .jpg, .jpeg, .png, .mov, .mp4, .wmv, .webm, .mpeg, .m4a, .wav, .mp3, and .ogg), scheduling features for appointments and cancellations, tracking a patient's attendance rates and reasons for cancellations (canceled by patient or provider, no show, absent, and the like) in a missed visit log, billing and payment options, integrated session planning tools to create detailed session plans for future or subsequent sessions or appointments tailored to the client's goals and interests, hosting for HIPAA-compliant teletherapy sessions, and the like. The input data and PHI related to the patient profile 210 are not depicted in FIG. 6.

The method 500a can continue with an operation 520a of recording, or operation 520b uploading a recording of, speech from a speaker, or more than one speakers, in an appointment or session between the patient and the provider. The speech can be directly recorded using the system 100, when equipped with an electronic device 108 that has either the internal microphone/recording device 412 or the external microphone/recording device 414. In a computer-implemented method, the processor 202 of the server 102 can cause the processor 402 of the electronic device 108 to initiate an audio recording of the speech during a session or appointment using its recording devices 412, 414. Alternatively, the processor 402 of the electronic device 108 may initiate an audio recording of the session or appointment by initiation of the user of the electronic device 108.

For the electronic device 108 without such components, the recording of the appointment can be done using another electronic device 108 with recording capabilities, and the recording can be uploaded to the system 100. In a computer-implemented method, the processor 202 of the server 102 can receive an uploaded audio file to the system 100 from another electronic device 108 or another server 102a-102n or from the database 106 via the network 104 and each device's respective communications units 206, 306, 406. The ability to upload a recording from the electronic device 108 may be preferable when appointments or sessions are conducted in a location with poor or no internet connection, so the electronic device 108 is not connected to the network 104 to access the system 100; in such cases, the recording is stored in the storage medium 404 of the electronic device 108 until a connection to the network 104 is restored such that the electronic device 108 can access the system. Recordings may be stored in the storage medium 204 of the database 106, either upon upload to the system 100 before it is processed, or after it is processed (especially after a certain number of days have passed for storage purposes (e.g. after 90 days)).

Speech originating from one or more speakers during an appointment or session may be recorded with standard audio-recording technology, such as either the internal microphone/recording device 412 or the external microphone/recording device 414, or using audiovisual means rather than just audio means. The audio recording can be associated with appointment data 416, such as the time, length, and date of the appointment; the provider who conducted the appointment; and any resulting documentation from the processing and analysis of the recording (such as a transcript, a provider note, objective scoring data, progress tracking, recommendations for future sessions, and the like). The audio recording may have a native file format, including at least one or more of .ogg, .mp3, .m4a, .wav, .mp4, .avi, .mov, and .m4v.

The method 500a can continue with the operation 530 of processing the audio file of the appointment or sessions. FIG. 5B illustrates a flowchart providing an exemplary embodiment of a set of sub-operations 500b to the operations 500a, focusing on the processing of an audio file recording of a therapy appointment, implementable in conjunction with the method of FIG. 5A and in accordance with aspects of the present disclosure.

The sub-operations 500b for processing the audio file can begin with the operation 532 of converting the audio file format, if necessary. This step may be optional if the native format of the recorded audio file is compatible with the system 100. In a computer-implemented method, the processor 302 of the server 102, or the processor 402 of the electronic device 108, can convert the file format of an incompatible audio file into a compatible file format (e.g., .ogg, .mp3, .m4a, .wav, .mp4, .avi, .mov, .m4v); in embodiments of the system 100 involving more than one server, another server 102a-102n may process the audio file, which can be sent using the communications unit 306 of the server 102 via the network 104. If the audio file is incompatible because it is too long for the system 100 to process, the audio file can be separated into different parts for processing, with the result of the processing combined.

The sub-operations 500b for processing the audio file can continue with an operation 534 of transcribing the words from the audio file to generate a time-stamped transcript. Timestamps within the transcript can correspond to each word. For a speech therapy patient focusing on articulation, the provider can configure the settings of the system 100 to transcribe the words spoke on at least a phoneme level. A phoneme is a basic unit of sound, more specifically the smallest unit of sound that may cause a change of meaning within a language but that doesn't have meaning by itself. For example, in the English word “ship,” there are three phonemes: “sh,” i,” and “p.” Such phoneme-level transcription and analysis may not be necessary for applications of this system 100 within occupational therapy, physical therapy, or non-speech-therapy medicine. The system 100 can be used within a healthcare or educational setting; educational settings can include a setting where the patient is the student and the provider is a teacher or a therapist employed by a school, or where student-therapists or student-providers are being trained.

The system 100 can be used with any language or dialect, so long as the processor 302 of the server 102, 102a-102n has been trained to process and analyze said language or dialect. If necessary, the processor 302 of the server 102, 102a-n can filter out background noise while generating the transcript. The contents of the transcript can be manually revised by a provider following the processing of the audio file (e.g. to correct an incorrectly transcribed word, or misplaced timestamp).

The sub-operations 500b for processing the audio file can continue with an operation 536 of diarizing the speakers from the audio file to separate out the transcribed words in the transcript based on whether each word was spoken by the patient (as the speaker) or the provider, or from any one or more other speakers. The transcript can be diarized by the processor 302 of the server 102 comparing the spoken words from the audio file with the sample of the patient's voice associated with the patient's profile 210 (retrieved by the processor 302 of the server 102, 102a-102n from the storage medium 204, 304 of the database 106 or server 102, 102a-102n using the communications units 206, 306 via the network 104). If the provider associated with the appointment data 416 or patient profile 210 also has a voice sample stored in the storage medium(s) 204, 304 of a component of the system 100, the processor 302 of the server 102 can also compare the spoken words from the audio file with the sample of the provider's voice. Using a sample of the provider's voice can be optional. Patient or provider voice samples can be around 30 seconds long, depending on the standardized sentence that is being read to produce the sample, and the speed of the speaker's speech. The diarization of the transcript can be manually revised by a provider following the processing of the audio file (e.g. to correct an incorrectly labeled speaker).

Speaker diarization may be optional if the recording only involves the patient (e.g., a patient submits a self-recorded homework session in which the patient is the only speaker, practicing exercises, activities, or other objectives away from the traditional therapy setting). Without being bound by theory, the system 100 can diarize up to 50 speakers within a group session; as more speakers are within an audio file, the importance of voice samples may increase.

Diarization can be important depending on the therapeutic context in which the system 100 is deployed. For example, in speech therapy, diarization ensures that when the transcript is later scored for errors, only the patient's words are being scored, and the provider's prompting, exemplar words, correction of the patient, or other spoken words are not scored with the patient's words, inflating the patient's score. On the other hand, in occupational or physical therapy contexts, diarization ensure that when a provider narrates the session and provides related feedback (e.g, “Patient is now drawing a circle. Great job! That one looks really good,” or “We are going to do five leg lifts, starting with your right leg. 1, 2—stay balanced —3 . . . looks like you're too tired to finish.”), it is easy to review the transcript to find the provider's feedback when scoring the related objective. Following diarization at operation 536, the sub-operation 500b can be completed, and the process returns to operation 540 within the overall operation 500a. The recorded audio file and related transcript is not depicted in, and further described in connection with, FIG. 6.

The operations 500a of evaluating audio recordings of patient speech can continue with operation 540 of analyzing the audio file. FIG. 5C illustrates a flowchart providing an exemplary embodiment of a set of sub-operations 500c to the operations 500a, focusing on the analysis of the audio file recording of a therapy appointment, implementable in conjunction with the method of FIG. 5A and in accordance with aspects of the present disclosure.

Sub-operations 500c can begin with an operation 542 of identifying at least one word from the transcript to be analyzed. The at least one word can be as little as one word or an entire phrase. Before operation 542, any PHI or other sensitive information from the transcript and any input data used by the system to analyze the transcript cam be redacted by the processor 302 of the server 102, 102a-102n, as well as any other actions relating to HIPAA or other legal compliance (this operation is not shown in FIG. 5C). The word can be identified by their timestamps from the transcript and correspond to an objective within the plan of care associated with the patient that was targeted in the session or appointment. A provider does not have to indicate which objective(s) associated with the patient's plan of care within the patient profile 210 was targeted within the appointment or session, instead the processor 302 of the server 102, 102a-102n can analyze the contents of the transcript, compare the contents against the objective(s) within a plan of care for the patient, and determine which objective(s) were targeted within the session or appointment based on what words were spoken throughout the transcript.

If the patient profile 210 does not include a plan of care or objective(s), the processor 302 of the server 102, 102a-102n can analyze the contents of the transcript to generate a broad visit note involving all detected phonemes across all positions, creating a less focused visit note than one that is evaluating specific objective(s) or goal(s).

Alternatively, the operation 542 may be preceded by operation 541 of generating an objective(s), if needed. The processor 302 of the server 102, 102a-102n can analyze the contents of the transcript to generate an objective(s) for which to analyze the audio file. Generated objective(s) can be made by comparing the contents of the transcript or provider note against a bank or library of objectives, goals, or resources stored within the library 212, stored on the storage medium 204, 304 of the database 106 or server(s) 102, 102a-n (retrieved by the processor 302 of the server 102, 102a-n from the storage medium 204, 304 of the database 106 or server 102, 102a-n using the communications units 206, 306 via the network 104). The processor 302 of the server 102, 102a-n can search the library 212 across horizon (short versus long term), discipline (speech, physical, occupational therapy, and the like), and major and minor focus areas.

For example, if the transcript or provider note discusses certain materials, answers, errors, scoring, and a summary of an activity, the processor 302 of the server 102, 102a-102n can search the library 212 to find matching objectives from the bank. As a non-limiting example, for a note that describes a material used as “Ultimate SLP around the world board game, what would you say? social communication targets Using functional social phrases in response to scenarios, max verbal & visual cues (modeling, role-play): can_i_play+-, conv++++++ topics: birthday, planning party at school, talking about highs school (where client will be, when he starts), ⅞=88%,” the processor 302 of the server 102, 102a-n can process this note to generate a goal such as “[Patient] will correctly follow directions; and understand and formulate sentences using appropriate markets of grammar (e.g., prepositions, pronouns, verb tense) and meaning (e.g., curriculum vocabulary, synonyms/antonyms, categories) with moderate cues, in 4 out of 5 opportunities (80% session accuracy) over three consecutive sessions,” with objectives related to this goal being “use functional social phrases in response to scenarios” scored at “⅞ (87.5%)” for this appointment or session. The patient's name can be inputted by the processor 302 of the server 102, 102a-n based on the information entered into the patient profile 210, but has been redacted here for privacy. The provider can revise the free-entry text of the goal, or number of opportunities or instances as the threshold level for the objective, depending on the level of the patient before importing or copying the goal from the goal bank and into the patient's plan of care associated with the patient profile 210. Goals for the patient can include a baseline and target percentage for non-articulation goals. This operation 541 is optional and can be skipped to proceed directly to operation 542 from operation 540 for patients who have an objective already entered into their plan of care as associated with their patient profile 210.

The library 212 of resources can include a goal or objective bank, in which reviewed and approved short-term and long-term objectives are uploaded to the library for insertion into a patient's plan of care. The library 212 can be searched by the processor 202, 302 of the component of the system 100 that the library 212 is stored on, based on tagging features. One tag, for example, can be only goals that the particular provider has written personally.

A series of non-limiting examples of objectives and significant words follows. For an articulation speech therapy patient, an exemplary objective or goal for correctly pronouncing phonemes can be “Correctly produce a /b/ sound during spontaneous conversation in different word positions”; exemplar words within the transcript can be identified by the processor 302 of the server 102, 102a-102n to include bass, cabin, and shrub, and the like. For a non-articulation speech therapy patient, an exemplary objective or goal can be “Use age-appropriate vocabulary to talk about the environment”; exemplar words within the transcript can be identified by the processor 302 of the server 102, 102a-102n to include a discussion about the patient's activities at school that day and upcoming weekend plans. For an occupational therapy patient, an exemplary objective or goal for performing an exercise or activity correctly can be “drawing a neat and complete circle 90% of the time in ⅘ trials over 3 therapy sessions”; exemplar words within the transcript can be identified by the processor 302 of the server 102, 102a-102n to include “circle” as well as feedback from the provider such as “good job” or “let's make the next one neater” or “the first one was not fully complete.” For a physical therapy patient, an exemplary objective or goal for performing an exercise or activity correctly can be “complete five leg lifts 90% of the time in 5/5 trials over 5 therapy sessions”; exemplar words within the transcript can be identified by the processor 302 of the server 102, 102a-102n to include numbers (of leg lifts completed) as well as feedback from the provider such as “Nicely done” or “stay balanced on the next one” or “you were unable to finish all five.” For use within the medical context, an exemplary objective for goal for determine whether a patient has a condition or diagnosis can be “determine whether patient has symptoms of diabetes”; exemplar words within the transcript can be identified by the processor 302 of the server 102, 102a-102n to include symptoms described by the patient or summarized by the provider, such as thirsty or excessive thirst, peeing or excessive urination, losing weight, hungry, fatigue or tired, and the like. More generalized objectives or goals can be whether a patient achieved an objective.

Sub-operations 500c can continue with an operation 544 of isolating audio fragments from the audio file by cutting short sound clips from the audio recording of the appointment or session. The content of the isolated fragments can include the previously identified significant words corresponding to an objective of the patient's plan of care. The processor 302 of the server 102, 102a-102n can use a set of timestamps within the transcript for any previously identified significant words to cut and save a sound clip of the significant word. For example, for the objective “Correctly produce a /b/ sound during spontaneous conversation in different word positions,” the processor 302 of the server 102, 102a-102n can cut a sound clip for each timestamp of the corresponding identified significant words within the transcript, such as bass, cabin, shrub, and the like.

As shown in FIG. 6, a sound clip of an identified significant word can be included in the report 600 resulting from an analysis of a therapy appointment produced by the method 500. The sound clip can be presented for playback using playback controls 604 within the report, so that the provider or patient can hear the sound clip of the identified significant word when reviewing the report results; the sound clip within FIG. 6 corresponds to the identified significant word “Bass” 606. The sound clip playback controls 604 may include buttons to press for playing, pausing, or stopping the sound clip, adjusting or muting the volume, and other features such as downloading the sound clip to the electronic device 108.

Such isolated fragments or sound clips can also be associated with the appointment data 416 and linked to the patient profile 210. Within the patient profile 210, a selection of sound clips may be selected and saved (or even combined into a longer audio file of an amalgamation of said sound clips) so that a patient may review particular successes or failures outside of their appointments or sessions, or a provider or patient can review a patient's progress for the same objective over time, across appointments or sessions; for some patients in which progress has been achieved, the processor 302 of the server 102, 102a-102n can assemble a highlight reel for the patient, presenting one sound clip from the beginning of the course of therapy, and another sound clip from the end of the course of therapy or once the objective has been met, to the patient via the portal for to the provider to review in a subsequent session to illustrate the patient's progress. The short, isolated fragments or sound clips can include a word or phrase.

Sub-operations 500c can continue with an operation 546 of evaluating the isolated fragments from the audio file to determine whether the objective of the plan of care for the patient was met. This determination can be based off a configurable threshold related to the objective for articulation-based speech therapy patients. The processor 302 of the server 102, 102a-102n can analyze and compare the objective(s) corresponding to the plan of care, associated with the patient and the patient profile 210 to determine whether an objective (generated by the server 102 or input by the provider) was targeted during the appointment, and whether that objective was met during the appointment.

For speech therapy patients focusing on articulation, the evaluation can include a scoring of whether an objective related to pronunciation was met. For example, as shown in FIG. 6, the report 600 shows that for the objective “Correctly produce a /b/ sound in the initial word position,” the processor 302 of the server 102, 102a-102n can total the number of instances that a word with a /b/ sound was spoken by the patient during the appointment, score the number of instances that the /b/ sound was correctly produced by the patient during the appointment against a configurable threshold, and then represent a score for that objective by calculating the number or percentage of correctly produced /b/ sounds out of total attempts (or trials) to produce a /b/ sound. The Objective Analysis Results section 618 of the report 600 in FIG. 6 shows that two objectives 620 were determined by the processor 302 of the server 102, 102a-102n to have been evaluated during this session (although as many objectives that were targeted during the session can be displayed in the report 600). In FIG. 6, each objective 620 is accompanied by information about the objective (optionally including its goal type and code, such as short-term (STG2) or long-term (LTG1)), such as level of prompting 626 and trials scoring information 622. For example, for the objective 620 concerning the /b/ phoneme, the patient required moderate prompting 626; out of 12 instances, the patient only correctly produced an /s/ sound above the threshold 6 times, for a trial score 622 of 6/12 or 50%.

For some speech therapy patients focusing on articulation of specific word(s) or phrases, rather than just sounds, the processor 302 of the server 102, 102a-102n can break each word in the phrase down into its distinct phonemes, and determine whether each phoneme in the word was correctly pronounced by repeating the above steps; for example, in the English word “bass,” there are three phonemes: “b,” “a,” and “s,” and the processor 302 of the server 102, 102a-102n can evaluate the pronunciation of each of the three phonemes to calculate a total pronunciation score for the word.

As shown in FIG. 6, the scoring of a word identified as significant can be included in the report 600 resulting from an analysis of a therapy appointment produced by the method 500. The significant word “Bass” 606 was evaluated for phoneme scoring in the word analysis results section 602 of the report 600. Within the exemplary section 602 of the exemplary report 600, only one identified significant word 606 is shown, but the word analysis results section 602 can include as many words that were identified as significant during the course of the appointment. Each individual instance of the patient saying the identified significant word “Bass” would have its own sound clip with playback controls 604 and word analysis results 602. In FIG. 6, the evaluation of the sound clip of the significant word “Bass” 606 is scored overall as “65” 608, accompanied by a color-coded score indicator 612 which can correspond to the configurable threshold designations for scoring (e.g., green is excellent between 90-100, orange is good between 70-90, red is poor between 0-70, and the like; for “Bass” 606, with a score of 65 608, the score indicator 612 would be displayed as a filled-in red circle). The evaluation of the significant word “Bass” 606 is also broken down into its individual phonemes 610 (/b/, /a/, /s/), which are each individually scored 614 with a numerical value and color-coded score indicator 612. For example, the patient corresponding to this exemplary excerpt of the report 600 has a phoneme score 614 of 62 for the /b/ phoneme 610 within this particular isolated sound clip of the identified significant word “bass.” The word analysis results 602 may also include a “sounds like” section 616, in which the processor 302 can notate an incorrect phoneme where the patient incorrectly used another sound in place of the /b/ sound, (e.g., “sounds like /d/”). For example, in FIG. 6, the patient's /b/ phoneme 610 in the scored identified significant word “bass” 606 sounded like a /d/ 616 within this particular isolated sound clip.

As shown in FIG. 6, the scoring of all phoneme(s) can be summarized in the speech sound chart section 628 of the report 600 resulting from an analysis of speech under the method 500. The speech sound chart section 628 may be limited to phoneme scoring of only words identified as significant in operation 542, or all phonemes from the transcript as a whole; FIG. 6 only displays a handful of exemplary scorings for various phonemes within the sound chart 628, but the chart can include as many phonemes as were scored within the appointment by the processor 302 of the server 102, 102a-102n.

Each scored phoneme 632 can have its individual summary section 630 within the sound chart 628 of the report 600. In each individual summary section 630, the phoneme 632 has pronunciation scores 638 with corresponding color-coded score indicators 612 for each different position (initial, middle, final) 634 within a word where the phoneme was said by the patient. For example, for the word “bass,” the /b/ phoneme is in the initial position; for the word “cabin,” the /b/ phoneme is in the middle position; and for the word “shrub,” the /b/ phoneme is in the final position. For each position 634, a count 636 of instances in which the phoneme was in that position in words during the session is displayed. For example, as shown in the phoneme summary 630 in FIG. 6, the patient has a combined pronunciation score 638 of 45 for the 10 instances 636 in which the patient used the /b/ phoneme 632 in the initial position 634.

The processor 302 of the server 102, 102a-102n can also evaluate the transcript, particularly the speech diarized to the provider, to determine the level of prompting (spontaneous, minimal, moderate, high) that the therapist had to give the patient before or during the period of time that the objective was being targeted during the assessment; in FIG. 6 of the report 600 from the analysis of an appointment using the method 500, the prompting level 626 is shown for each objective within the objectives and trials section 618 of the report (e.g., the patient required a moderate level of prompting for the objective about correctly producing a /b/ sound in the initial position). Where needed, in FIG. 6 of the report 600, the editing tools 624 for revising the trials information scoring the objective are shown. Using the editing tools 624, the provider can edit the trials information if improperly scored by the processor 302 or delete the objective 620 from the report 600 altogether.

For speech therapy patients that are not targeting articulation, occupational therapy patients, physical therapy patients, and an application of the system 100 in a generalized medical context, the processor 302 of the server 102, 102a-102n does not automatically score the transcript's identified significant words or phrases, but instead prompts the provider to manually enter a score for the objectives. However, the processor 302 of the server 102, 102a-102n can identify significant words related to the objective(s) targeted in the appointment or session, and count the number of instances the objective(s) were tested, and interpret the transcript to determine whether each instance was successful or not. For example, for the non-articulation speech therapy objective or goal of “[u]se age-appropriate vocabulary to talk about the environment,” the processor 302 can identify instances of age-appropriate vocabulary (based on the age of the patient as per the patient profile 210) and compare it against the number of instances of age-inappropriate vocabulary and the therapist can input the total the number of instances that the patient used inappropriate vocabulary and appropriate vocabulary (for example, in FIG. 6 within the trials section 622 of the objective analysis results section 618 of the analysis report 600), so the processor 302 of the server 102, 102a-102n can then calculate a score for that objective by calculating the number or percentage of appropriate vocabulary out of total instances of vocabulary usage concerning the environment. This process is similar for occupational therapy and physical therapy patients and their objectives (identifying instances of an activity or exercise or other achievement, and identifying words or phrases indicating the level of success for each instance, and then totaling instances of correct versus incorrect performance of an activity or exercise or other achievement of the objective to calculate a percentage score). For example, in FIG. 6 of the report 600, had one of the displayed objectives within the objectives and trials section 618 been a non-articulation speech, occupational, or physical therapy objective, such as completing five leg lifts, the processor 302 would have identified each instance of a leg lift narrated by the therapist in the transcript (e.g., “That's one leg lift.”), and identified whether each instance was successful based on the narrated feedback of the therapist in the transcript (e.g., “Good job!” or “Not quite.”), before prompting the provider to input such information into the trials section 622 (e.g., if the transcript revealed only three successful leg lifts, the provider can input 3/5 as trial information). The provider can use the editing tools 624 to input, or correct, the trials information 622 (i.e., the correct instances out of the incorrect instances, for the processor 302 to calculate the revised percentage).

For an application of the system 100 to a general medical context, for example, diagnosing whether a patient has particular condition or disease (e.g. diabetes), the provider can review the time-stamped transcript to total the number of symptoms that the patient described during the appointment that indicate a certain condition against the known symptoms for that condition to determine whether a patient has enough symptoms for that condition to apply to the patient.

The above processes can be done for each instance of the objective(s) being tested in the appointment or the session and for each of the objective(s) targeted in the appointment or the session. Following evaluation at operation 546, the sub-operation 500c can be completed, and the process returns to operation 550 within the overall operations 500a.

The operations 500a of evaluating audio recordings of patient speech can continue with operation 550 of drafting post-appointment documentation based at least in part on an analysis of the audio file (as completed in operations 540-546). FIG. 5D illustrates a flowchart providing an exemplary embodiment of a set of sub-operations 500d to the operations 500a to draft post-appointment documentation based off an audio file recording of a therapy appointment, implementable in conjunction with the method of FIG. 5A and in accordance with aspects of the present disclosure.

Sub-operations 500c can begin with operation 552 of generating a provider visit note corresponding to the appointment or session. The provider note can be generated by the processor 302 of the server 102, 102a-102n based on the generated transcript of the audio file associated with the appointment data 416, any objective(s) or goal(s) that may be present in the patient profile 210, and the error analysis completed in operation 546.

The processor 302 of the server 102, 102a-102n can generate a provider note in SOAP format, a common format for provider notes in the therapy context; however, both of the above exemplary prompts can be revised to generate a note that is not in SOAP format. When generating a SOAP-formatted provider note, the processor 302 of the server 102, 102a-102n can generate each subsection of the SOAP note based on an analysis of the transcript; a non-limiting example will follow, in which a provider note was generated based off a transcript of an appointment in which a provider used an activity concerning sea creatures to target an objective relating to the production of the /b/ sound in an initial position (an articulation speech therapy patient). An exemplary generated portion of a provider note for the “subjective” field may include: “The patient was able to name a variety of sea creatures during the session. The patient was able to name the sea creatures with minimal prompting.”

An exemplary portion of a provider note for the “objective” field may include: “During the session, the patient focused on articulating words with initial /b/ sounds, primarily through naming a variety of sea creatures. Achievements are qualified as follows: in trials with words starting with ‘b’, the patient achieved 50% success pronouncing ‘bream,’ ‘barracuda,’ ‘beluga whale,’ and ‘bass.’ There were varying degrees of success with other sea creature names that do not begin with the target sound, providing an opportunity for continued therapy. The aim for future sessions is to improve proficiency in articulating words that start with /b/ sounds.” The “objective” section of the note can also include an “objectives/trial” section, in which the name of an objective or goal, and the scoring from operation 546 can be summarized in the note. An example of this section 618 is displayed in FIG. 6; for example, the objective 620 is to correctly pronounce the /b/ phoneme in the initial position; Goal: LTG1→STG2, which required moderate (“Mod”) prompting 626 with a trial score 622 of 6/12 (50%). Multiple objectives may be targeted in a session and summarized within this section of the provider note. The “objective” section of the note can also include a “Speech sound chart” 628 in which each identified phoneme 632 and its error score from the transcript can be presented in a summary 630 for that phoneme, with total number of instances 636 with various positions 634 and an overall score 638 for each position 634 of that phoneme for that session or appointment. As shown in FIG. 6, even phonemes 632 outside of the objectives 620 for the appointment, (e.g., the /f/ phoneme) may be displayed in the sound summary chart 628. Again, for non-articulation speech therapy, occupational therapy, physical therapy, and other medical contexts, the provider may have to manually input the prompting 626 or trials 622 information for each objective 620 in the objective analysis results section 618 of the appointment report 600.

An exemplary portion of a provider note for the “assessment” field may include: “The patient is making progress towards his short-term goals. He is able to correct produce the /b/ phonemes in the initial word position correctly with 50% accuracy. However, he still needs to improve his articulation skills to reach the goal of 80% accuracy for the /b/ phoneme in the initial word position.” Again, multiple objectives may be targeted in a session and summarized within this section of the provider note.

An exemplary portion of a provider note for the “Plan” field may include: “Continue to work on the patient's articulation skills, focusing on the /b/ phoneme. Incorporate more words with these phonemes in the initial position into the therapy sessions. Monitor the patient's progress and adjust the therapy plan as needed.”

If there are no objectives associated with a patient's plan of care, the processor 302 can engage in operation 553 of generating the objectives for use in drafting a visit note.

For a SOAP note in a patient that has no goals entered into the plan of care, a goal may be generated in the operation 553 by the processor 302 for a patient's plan of care associated with the patient profile 210 by analyzing the “objective” section of the generated visit note. This step 553 is optional and can be skipped to proceed directly to operation 554 from operation 552 for patients that have goals entered into their plan of care associated with their patient profile 210.

Provider notes are drafted primarily as a document for the provider (and not the patient; a summarization of the provider note for the patient or their family can be done for the patient as discussed below in operation 546). The provider note can also be referenced or reviewed in an insurance audit, hence the focus on goals and therapy activities within it. The processor 302 of the server 102 may also populate other information from the appointment data 416, such as the date and time of the appointment, the associated provider(s) who ran the appointment and/or will sign off on the note, and the like. The processor 302 of the server 102 may also populate other information from the patient profile 210, such as the type of therapy discipline that the patient is participating in (speech, occupational, physical, general medicine), and information from the plan of care (including the objectives and goals).

Sub-operations 500c can continue with operation 554 of editing or revising the generated visit note, as needed and if necessary. The processor 302 of the server 102, 102a-102n may prompt the provider to review and revise the generated provider note as necessary before signing the note for entry into an EMR system. As shown in FIG. 6, even without such a prompt, the provider can use editing tools 624 to revise the trial counts 622. Revising the generated note can include loading portions of a prior note, or using templates or other saved phrases or keyboard shortcuts. Revising the generated note can also include the provider prompting the processor 302 of the server 102, 102a-102n to perform the error analysis on other phonemes, words, or phrases. Allowing for provider revisions to any output of the system 100 can provide for flexibility and accuracy of the resulting output. This operation 554 is optional and can be skipped to proceed directly to operation 556 from operation 552, 553 for providers that do not wish to revise or edit the generated visit note.

The generated (and revised, if necessary) provider note can be entered into the EMR system as part of the record for the appointment or session; if the provider does not use the EMR of the patient profile 210 within the system 100 as stored on the storage medium 204, 304 of the database 106 or server(s) 102, 102a-102n, the provider can export the generated visit note as a plan text file, which may require the use of an API to import into another system.

Sub-operation 500c can continue with operation 556 of summarizing the visit note (as written for a provider) for consumption by a patient (or their family, as done in the cases of a minor patient). In summarizing the provider note, the processor 302 of the server 102 can simplify the provider note by removing or explaining abbreviations and removing sections pertaining to in-depth scoring and speech sound chart(s), as well as revising clinical language to laymen's terms. Continuing the non-limiting example discussed in the SOAP note above, an exemplary patient summary of the note can be: “In the therapy session, [Patient] engaged in conversations about sea creatures. [Patient] showed good progress with the /b/ sound, reaching 50% accuracy. However, challenges were noted with the/b/sound in ‘bass.’ The plan involves continued practice with challenging words, using structured conversations and various activities to improve pronunciation in these areas.” As with all previous examples, the summary may involve multiple objectives, so long as the provider note involved multiple objectives. The patient summary can be uploaded to a patient portal connected to the system 100 or otherwise directly transmitted to the patient, their parent(s)/guardian(s), and other non-professional stakeholders (with client/parental consent, such as teachers). This generation, uploading, and transmitting of a patient-friendly summary of EMR data constitutes an improvement in patient communication systems. This operation 556 is optional and can be skipped to proceed directly to operation 560 from operation 554 for providers that do not wish to generate such a summary for their patients. Following summarization at operation 556, the sub-operations 500d can be completed, and the process returns to operation 560 within the operations 500a.

The operations 500a of evaluating audio recordings of patient speech can continue with operation 560 of tracking a progression of the patent within the plan of care. FIG. 5E illustrates a flowchart providing an exemplary embodiment of a set of sub-operations 500e to the operations 500a to track a progression of a patient across appointments based off an audio file recording of a therapy appointment, implementable in conjunction with the method of FIG. 5A and in accordance with aspects of the present disclosure.

Sub-operations 500e can begin with operation 562 of extracting performance information from the analysis of the audio file (operations 540-546) and the generated post-appointment documentation (operations 550-556) corresponding to the objectives from the plan of care for the patient as targeted in the appointment (using the appointment data 416 where necessary). The processor 302 of the server 102, 102a-102n can use the error analysis and/or generated transcript to produce progress tracking data related to the objective(s) or goal(s) in the patient's plan of care associated with their patient profile 210 by comparing the objective(s) or goal(s) in the patient's plan of care with the analysis results from operations 540-546. In this way, performance information data may include the error scoring 608, 638 generated by the system 100 and/or trial data 622 input by the provider for non-articulation speech therapy patients in operations 546, 552-554, such that the extracted performance information can be used to determine whether and to what extent the objective was met during the appointment. In articulation speech therapy patients, progress tracking can be down to the phoneme level, and summarized by phoneme in a speech sound chart 628. For example, in continuing the above-mentioned non-limiting example as shown in FIG. 6 about the appointment in which the provider is targeting the /b/ sound, the extracted performance information relating to the objective 620 of correctly producing of the /b/ sound in an initial position can be the pronunciation error score of 6/12 (50%) 622. In another non-limiting example where an objective was to “[u]se age-appropriate vocabulary to talk about the environment,” the extracted performance information relating to the objective can be the trial information input by the provider of six age-appropriate vocabulary uses versus two non-age-appropriate vocabulary uses, totaling eight instances, or 6/8 trials (75%).

The extracted performance information data, as related to at least one objective from the plan of care for the patient can be used to determine whether and to what extent the objective was met during the appointment. In the same non-limiting examples set forth above, if the configurable threshold for the objective of using age-appropriate vocabulary was 70%, then the objective was met during an appointment where the extracted performance information data indicated a score of 75% (greater than the threshold). In another non-limiting example, if the configurable threshold for the objective of correctly producing the /b/ sound was 70%, then the objective was not met during an appointment where the extracted performance information data indicated a score of 50% (less than the threshold).

Sub-operations 500e can continue with operation 564 of comparing the extracted performance information from operation 562 to previous performance information from prior appointments or scheduled to determine the change in the performance information across previous appointment(s), for at least two appointments (including the current appointment) across time. The processor 302 of the server 102, 102a-102n can access such previous performance information associated with the patient profile 210 (retrieved by the processor 302 of the server 102, 102a-102n from the storage medium 204, 304 of the database 106 or server 102, 102a-102n using the communications units 206, 306 via the network 104). For example, in continuing the above-mentioned non-limiting example about the appointment in which the provider is targeting the usage of age-appropriate vocabulary to discuss the environment, the processor would compare the extracted performance information from the appointment ( 6/8 trials (75%)) with extracted performance information for the same objective from a previous appointment (e.g., ⅜ trials (37.5%)), showing an increase in progress over time given the increased number of correct trials and corresponding percentage.

To track a patient's progression within their plan of care over time, wherein providers can differently word objectives or apply the same objective across different therapy activities and other related performance information across sessions, the processor 302 of the server 102, 102a-102n can classify each activity to then aggregate similar activity data together to ensure that all instances of performance information for the same objective is extracted and compared over all appointments in time associated with the patient via the patient profile 210.

Sub-operation 500e can continue with operation 566 of summarizing performance information to represent the determination of whether and to what extent the objective was met by the patient across at least two appointments in time. The summary can be in a report or visually represented in a generated chart or graph (e.g., line graph) for both short-and long-term goals. The processor 302 of the server 102, 102a-102n can use the extracted performance information data from the appointment (based on the appointment data 416 and the visit note generated in operation 552) and previous appointments (retrieved by the processor 302 of the server 102, 102a-102n from the storage medium 204, 304 of the database 106 or server 102, 102a-n using the communications units 206, 306 via the network 104) to generate either a summary report or visual representation of the extracted performance information data over time.

A visual summary in a line graph can include a time progression of each scored instance or trial data across time, and can also include the patient's baseline level related to the objective (at first testing) and the target or threshold level desired to meet the objective. Continuing the above non-limiting example, given the extracted performance information from the appointment ( 6/8 trials (75%)) and the extracted performance information for the same objective from a previous appointment (e.g., ⅜ trials (37.5%)), a visual representation of this comparison can be summarized in a line graph (with an x-axis of time, and a y-axis of percentage of correct trials from 0% to 100%) with at least two points: a first point for the date of the previous appointment at 37.5%, and a second point point to the left of the first point for the date of current appointment at 75%, showing an increase over time from the first point to the second point given the increased number of correct trials and corresponding percentage. The line graph may also have lines representing a mean, median, baseline, or threshold level relating to the objective. Other visual representations of data can be used other than a line graph, such as charts, other graphs, or other pictorial representations of performance data.

In articulation speech therapy patients, where progress tracking can be down to the phoneme level, performance information related to the pronunciation error analysis scores related to the phoneme can be extracted and aggregated across time to present a summary report of “Articulation Progress” associated with the patient profile 210 of the patient; the Articulation Progress report can indicate to a provider which phonemes (and in which position) the patient needs to work on in subsequent sessions by showing scores for any phoneme that was scored in any appointment associated with the patient profile 210, and for each scored phoneme across various appointments over time (whether or not associated with an objective), even in different positions, display scoring information and corresponding isolated fragments of audio. For example, an Articulation Progress report for a patient the/b/phoneme can show a listing of each instance in which the/b/phoneme was scored by the processor 302 of the server 102, 102a-102n in operation 546 for any previous appointment(s) (listed by date of the appointment), the corresponding word for each listing (e.g., “bass”), the position of the phoneme within the word (e.g., “initial” for the word “bass,” as the/b/sound is in the first position), the corresponding score for each instance (e.g. 1-100), and an isolated fragment or sound clip of audio of the patient speaking the word during that appointment. In addition, in articulation speech therapy patients, where progress tracking can be down to the phoneme level, performance information related to the pronunciation error analysis scores for each phoneme can be aggregated and analyzed across time to show the scores of each instance of the patient producing the phoneme across various appointments or sessions, including an average and/or median calculated score based on the aggregated scores for the examined appointments. Other types of summary reports can be generated from the same performance information or data.

In this way, between operations 540 of analyzing an audio file, including evaluation of audio fragments against an objective in operations 546 and 560-566 of tracking a progression of a patient across time, the system 100 receives information in the form of an audio recording (with no other user/provider input), and then dissects and analyzes that information to produce new data (related to scoring an objective) to generate the report 600 or notification concerning the patient's performance within an appointment and tracking said performance across appointments in time. Furthermore, the use of the system 100 to generate visit notes, phoneme scoring 630, objective trials/scoring 622, and progression tracking can improve accuracy in provider notes and reports. The integration of both patient interest(s) and goal(s) or objective(s) in the outputs of the system 100 allows for efficient, personalized, and goal-directed therapy planning based on up-to-date references, resources, and activities.

In some embodiments of the system 100, the provider can input two dates to limit the date range that the processor 302 of the server 102, 102a-102n can search for performance information in order to summarize or visually represent only appointments that fall within a certain timeframe (between two input dates) rather than the entire history of the patient's appointments associated with the patient profile 210.

Following summarization at operation 566, the sub-operations 500e can be completed, and the process returns to operation 570 within the overall operations 500a.

The operations 500a of evaluating audio recordings of patient speech can continue with operation 570 of creating recommendations for a subsequent appointment based on the analysis of the audio file and the plan of care for the patient. FIG. 5F illustrates a flowchart providing an exemplary embodiment of a set of sub-operations 500f to the method 500a to create recommendations for a subsequent appointment(s) based off an audio file recording of a therapy appointment, implementable in conjunction with the method of FIG. 5A and in accordance with aspects of the present disclosure.

Sub-operation 500f can begin with operation 572 of drafting a treatment plan for the subsequent appointment or session for the patient. This may relate to the “plan” section of a SOAP-formatted visit note generated in operation 552, representing the objective(s) within the plan of care that were targeted by the provider during the current appointment. To draft the treatment plan, the processor 302 of the server 102, 102a-n can retrieve information from the storage medium 204, 304 of the database 106, server 102, or another server 102a-102n, including the transcript generated in operations 534-536, the visit note generated in operation 552, and other information from the patient profile 210 (including at least the patient's interests (from the input data) and the objective(s) associated with the plan of care of the patient).

Sub-operation 500f can continue with operation 574 of searching the library 212 for recommended resources or references to inform the treatment plan and/or activity list(s) for the subsequent appointment. The processor 302 of the server 102, 102a-102n can perform a word vector search of word-embedding data from the metadata of resources within the library 212 to search for resources that correspond to the objective(s) within the patient's plan of care. The metadata of the resources within the library 212 can include who created and/or uploaded the resource to the library 212, the strategy or technique involved, the objective(s) targeted, and other taxonomic information related to the resource. Resources can include, as an example, PDF files of various clip-art or other images to prompt the patient into working on various phonemes during activities without prompting from the therapist; for example, to work on the /b/ phoneme, an activity involving naming all sea creatures with the /b/ sound in the initial position can include photographs of sea creatures with the /b/ sound in the initial position to prompt the patient to initiate the words, rather than the therapist saying the desired words to get the patient to repeat the /b/ sound).

The resources in the library 212 can also include reference links to evidence-based practice research or scientific publications, searched within the library 212 by the processor 302 of the server 102, 102a-102n based on queries (related to the transcript of the session or objective(s) in the care of plan for the patient) of the library meant to search the tagged information for the reference, such as the word-embedding data from the library 212 that have been categorized with similar meta data as discussed above.

Sub-operation 500f can continue with operation 576 of searching the library 212 for recommended activities to inform the treatment plan and/or activity list(s) for the subsequent appointment. The processor 302 of the server 102, 102a-102n can perform a word vector search of word-embedding data from the metadata of activities within the library 212 to search for activities that correspond to the objective(s) within the patient's plan of care. The metadata of the activities within the library 212 can include who created and/or uploaded the activity to the library 212, the strategy or technique involved, the objective(s) targeted, and other taxonomic information related to the activity (e.g., whether the activity concerns expressive language objective(s) or articulation objective(s), whether the objective(s) are a major or minor focus of the activity, and the like).

Sub-operation 500f can continue with operation 578 of the processor 302 of the server 102, 102a-n prompting the provider to adapt the plan drafted in operation 572 based on the aforementioned resources, references, and/or activities found in operations 572-573. This step 578 is optional and can be skipped for providers that do not wish to adapt the treatment plan. For example, where appropriate or desired activities cannot be found within the library 212, the processor of the server 102, 102a-n can generate activity lists to target a described objective (e.g., “use age-appropriate vocabulary to spontaneously describe skills”) based on a prompt (e.g., “What are the steps for riding a bike?”). Following adapting at step 578, the sub-operation 500f can be completed, and the process returns to the end of the overall operations 500a.

The information displayed in a report 600 may be customizable based on the preferences of the user-provider using the system 100, the input data from the provider, and/or the goals or objectives of a patient's plan of care. For example, for a non-articulation speech therapy patient, or patients in occupational therapy, physical therapy, and other general medical contexts, the word analysis results section 602 and the speech sound chart 628, both based on phoneme pronunciation scoring of an articulation-focused patient, may not be included, and other sections more relevant to such disciplines may be included instead. For example, without being bound by theory, the system 100 may analyze and score vocabulary levels, if a speech patient were working on an exemplary objective, such as “using age-appropriate vocabulary.” In another example, without being bound by theory, the system 100 may analyze and score patient performance on non-speech therapy activities based on pre-set scoring words or phrases from the provider, such as assigning a 1/5 for “try again,” a 2/5 for “not quite,” a 3/5 for “good,” a 4/5 for “great,” a 5/5 for “excellent,” or other similar scoring phrases that can be pre-programmed into the storage medium 204, 304 of the database 106 or server 102, 102a-102n for the processor 302 of the server 102, 102a-102n to execute while scoring. The report 600 can also include a selection of sound clips from various appointments or sessions over time as a highlight reel to illustrate the patient's progress over the course of therapy by presenting one sound clip from the beginning of the course of therapy, another sound clip in the middle of the course of therapy, and another sound clip from the end of the course of therapy or once the objective has been met (or alternatively at other patient milestones, such as the patient getting older at four years old, five years old, and six years old, for example). The report 600 may also include other progress tracking information for the objective(s), goals(s), or various phoneme scoring for a patient over time across sessions or appointment; for example, summary tables or charts, graphs or other visual or non-visual representations of such data.

The present embodiments are not restricted by the architecture of the aforementioned components, so long as the components, whether directly or indirectly, supports the operations as described herein. For example, one or more operations 500a, and the sub-operations 500b-500f, may be carried out by the processor 402 of one or more electronic devices 108, and information or data may be retrievably accessed or stored on the storage medium 404 of the one or more electronic devices 108. The embodiments of the method 500, as described herein, are implemented as logical operations performed by the system 100. The logical operations of these various embodiments of the present disclosure are implemented (1) as a sequence of computer implemented steps or program modules running on a computing system and/or (2) as interconnected machine modules or hardware logic within the computing system. The implementation is a matter of choice dependent on the performance requirements of the computing system implementing the disclosure. Accordingly, the logical operations making up the embodiments of the disclosure described herein can be variously referred to as operations, steps, or modules. As such, persons of ordinary skill in the art may utilize any number of suitable electronic devices and similar structures capable of executing a sequence of logical operations according to the described embodiments.

To facilitate the understanding of the embodiments described herein, a number of terms have been defined above. The terms defined herein have meanings as commonly understood by a person of ordinary skill in the areas relevant to the present disclosure. The terminology herein is used to describe specific embodiments of the disclosure, but their usage does not delimit the disclosure, except as set forth in the claims.

The examples are provided above are used to facilitate a more complete understanding of the disclosure. The preceding examples illustrate the exemplary modes of practicing the disclosure. However, the scope of the disclosure is not limited to specific embodiments disclosed in these examples, which are for purposes of illustration only, since alternative methods can be utilized to obtain similar results.

Those skilled in the art will recognize, or be able to ascertain, using no more than routine experimentation, numerous equivalents to the specific substances and procedures described herein. Such equivalents are considered to be within the scope of this disclosure and are covered by the following claims.

What is claimed is:

1. A method for evaluating word structures in patient speech, the method comprising:
recording speech originating from one or more speakers to create an audio file, wherein the audio file has a native file format associated therewith;
transcribing the audio file to generate a transcript that identifies one or more words in the speech originating from the one or more speakers;
detecting one or more phonemes in the one or more words in the speech originating from the one or more speakers, wherein each of the one or more phonemes correspond to a sound-based unit in a position in each of the one or more words;
isolating the audio file into one or more audio fragments that correspond to the one or more phonemes in the one or more words;
analyzing the one or more audio fragments to determine whether the one or more phonemes in the one or more words was pronounced correctly or incorrectly in the corresponding sound-based unit in the position in the one or more words;
scoring performance of the speech originating from the one or more speakers by calculating correct and incorrect pronunciations of the one or more phonemes in each of the one or more words; and
extracting a success rate for the correct pronunciations as compared to the incorrect pronunciations for the one or more phonemes as with respect to the corresponding sound-based unit in the position in the one or more words to generate an objective targeting the one or more phonemes for subsequent performance of the speech originating from the one or more speakers for the purpose of tracking said subsequent performance.

2. The method of claim 1, wherein:
the audio file has speech from more than one of the one or more speakers.

3. The method of claim 2, further comprising:
diarizing the speech in the audio file to separately identify on the transcript which of the one or more speakers originated the one or more words in the speech.

4. The method of claim 1, wherein:
the transcript has one or more timestamps associated therewith, each of the one or more timestamps corresponding to each of the one or more words in the speech originating from the one or more speakers.

5. The method of claim 1, wherein:
the position of the sound-based unit in each of the one or more words constitutes an initial position, a middle position, or a final position of the one or more phonemes.

6. The method of claim 1, wherein:
the native file format associated with the audio file is any one of .ogg, .mp3, .m4a, .wav, .mp4, .avi, .mov, and .m4v.

7. The method of claim 1, wherein:
the objective for subsequent performance of the speech originating from the one or more speakers constitutes a configurable threshold for correct pronunciations for the one or more phonemes as with respect to the corresponding sound-based unit in a position in the one or more words.

8. A system for evaluating word structures in patient speech, the system comprising:
a network;
an electronic device associated with one or more speakers, the electronic device having an audio-recording device connected thereto and further including a communications unit allowing for communicative coupling to the network;
a server having a communications unit for communication with the electronic device via the network, the server having a processor configured to execute instructions residing on a storage medium and configured to:
create an audio file based upon speech recorded by the audio-recording device connected to the electronic device, wherein the audio file has a native file format associated therewith;
transcribe the audio file to generate a transcript that identifies one or more words in the speech originating from one or more speakers;
detect one or more phonemes in the one or more words in the speech originating from the one or more speakers, wherein the one or more phonemes correspond to a sound-based unit in a position in the one or more words;
isolate the audio file into one or more audio fragments that correspond to the one or more phonemes in the one or more words;
analyze the one or more audio fragments to determine whether the one or more phonemes in the one or more words was pronounced correctly or incorrectly by the one or more speakers in the corresponding sound-based unit in the position in the one or more words;
score performance of the speech originating from the one or more speakers by calculating correct and incorrect pronunciations of the one or more phonemes in the one or more words; and
extract a success rate for the correct pronunciations as compared to the incorrect pronunciations for the one or more phonemes as with respect to the corresponding sound-based unit in the position in the one or more words to generate an objective targeting the one or more phonemes for subsequent performance of the speech originating from the one or more speakers for the purpose of tracking said subsequent performance.

9. The system of claim 8, wherein:
the audio file has speech from more than one of the one or more speakers; and
the server is further configured to diarize the speech in the audio file to separately identify on the transcript which of the one or more speakers originated the one or more words in the speech.

10. The system of claim 8, wherein:
the transcript has one or more timestamps associated therewith, each of the one or more timestamps corresponding to each of the one or more words in the speech originating from the one or more speakers.

11. The system of claim 8, wherein:
the objective for subsequent performance of the speech originating from the one or more speakers constitutes a configurable threshold for correct pronunciations for the one more phonemes as with respect to the corresponding sound-based unit in the position in the one or more words.

12. A method for evaluating word structures in patient speech, the method comprising:
inputting data corresponding to a profile of a speaker, such data including at least a configurable threshold for correct and incorrect pronunciations of one or more phonemes;
recording speech from the speaker to create an audio file, wherein the audio file has a native file format associated therewith;
generating a transcript that identifies one or more words in the speech from the speaker, such transcript bearing timestamps corresponding to each of the one or more words;
dissecting the audio file into one or more audio fragments to target one or more phonemes in the one or more words, wherein the one or more phonemes correspond to a sound-based unit in a position in the one or more words;
determining whether the one or more phonemes in the one or more words was pronounced correctly or incorrectly by the speaker in the corresponding sound-based unit in the position in each of the one or more words;
scoring performance of the speech of the speaker by calculating correct and incorrect pronunciations of the one or more phonemes in the one or more words;
performing error analysis of the performance of the speech by comparing the correct and incorrect pronunciations of the one or more phonemes in the one or more words against the configurable threshold to determine a success rate for the correct pronunciations for the one more phonemes as with respect to the sound-based unit in the position in the one or more words; and
creating an objective for subsequent performance of the speech based upon the error analysis, the objective for subsequent performance prescribing an updated configurable threshold for correct and incorrect pronunciations for the one more phonemes as with respect to the corresponding sound-based unit in the position.

13. The method of claim 12, wherein:
the audio file has speech from one or more speakers other than the speaker.

14. The method of claim 13, further comprising:
diarizing the speech of the speaker from the speech of the one or more speakers other than the speaker in the audio file by separately identifying on the transcript which of the one or more speakers and the speaker originated the one or more words in the speech; and
comparing the one or more words in the speech from the one or more speakers and the speaker with a sampled audio recording of speech attributable to the speaker, the sampled audio recording being included in the data corresponding to the profile of the speaker.

15. The method of claim 12, wherein:
creating the objective for subsequent performance of the speech based upon the error analysis further comprises comparing the one or more words of the transcript against a bank of resources comprising word-embedded metadata.

16. The method of claim 15, wherein:
comparing the one or more words of the transcript against a bank of resources comprising word-embedded metadata further comprises performing a word vector search of the word-embedded metadata to identify other one or more words having the one or more phonemes pronounced incorrectly by the speaker.

17. The method of claim 12, wherein:
creating the objective for subsequent performance of the speech based upon the error analysis further comprises analyzing correct pronunciations for the one or more phonemes in the one more words in a corresponding first sound-based position against incorrect pronunciations for the one or more phonemes in the one or more words in a corresponding second sound-based position.

18. The method of claim 12, wherein:
scoring performance of the speech by calculating correct and incorrect pronunciations of the one or more phonemes in the one or more words depends upon the pronunciation of the corresponding sound-based unit in the position in the one or more words.

19. The method of claim 18, wherein:
the position of the sound-based unit in the one or more words constitutes an initial position, a middle position, or a final position of the one or more phonemes.

20. The method of claim 19, wherein:
scoring performance of the speech by calculating correct and incorrect pronunciations of the one or more phonemes in each of the one or more words is limited to a single sound-based unit in a single position in each of the one or more words, such single position of the single sound-based unit being one of the initial position, middle position, or the final position of the one or more phonemes.
